(19) United States
(12) Patent Application Publication
(10) Pub. No.: US 2023/0317272 A1
(43) Pub. Date: Oct. 5, 2023

(54) SYSTEMS AND METHODS FOR ORTHODONTIC TREATMENT PLANNING USING UNIVERSAL COMMANDS AND PROTOCOLS USING THOSE COMMANDS

(71) Applicant: Ormco Corporation (Spark/Aligner Division), Brea, CA (US)

(72) Inventors: Evan Yifeng Tsai, Rancho Cucamonga, CA (US); Asad Abu-Tarif, Irvine, CA (US); Karol Miranda, Tustin, CA (US); Jose Franco, Guanacaste (CR)

(21) Appl. No.: 17/711,279

(22) Filed: Apr. 1, 2022

(51) Int. Cl. G16H 40/67; G16H 20/30; G16H 30/20

(52) U.S. Cl. CPC G16H 40/67; G16H 20/30; G16H 30/20

(57) ABSTRACT

A system for orthodontic treatment planning. The system comprises a processor and memory. Computer-program instructions cause the system to display a user interface on a display. The user interface is for a user to input a prescription for treatment of the patient. The user interface provides a plurality of commands for selection. Each command is a one-word instruction, a two-word instruction, or a three-word instruction based on orthodontic nomenclature. The system receives selected two or more of the plurality of commands into the prescription for treatment. The two or more selected commands are to be applied according to a predetermined protocol to the patient's teeth. A database is configured to receive the prescription for treatment of the patient and to contain a plurality of other prescriptions for treatment of other patients. The database is configured to receive a plurality of other prescriptions from a plurality of users of the system.

### TECHNICAL FIELD

The present invention relates generally to the field of orthodontic treatment and, more particularly, to systems and methods for orthodontic treatment planning.

### BACKGROUND

Orthodontics is the practice of manipulating teeth to correct malocclusions between the teeth of the upper and lower dental arches. Typically, treatment of malocclusions includes the use of an orthodontic appliance that applies corrective forces to the teeth. Over time, these corrective forces coerce the teeth to move into their orthodontically correct positions.

One way of applying corrective forces to teeth is an orthodontic appliance referred to as an “aligner.” Other orthodontic appliances include orthodontic brackets that are secured to the teeth and are usable with an orthodontic archwire to apply corrective forces to a patient's teeth.

Aligners are generally supplied as a series of removable appliances that incrementally reposition the patient's teeth from their initial orientation, in which the teeth may be maloccluded, to their orthodontically correct and aesthetic orientation. Patients being treated with aligners can insert and remove the aligners at will, and therefore do not need to visit the orthodontist to advance their treatment. Rather, when the currently worn aligner has moved the teeth to at or near a final orientation for that aligner, the patient merely begins using the next aligner in the series according to a treatment plan. In that regard, each aligner in the series differs from all other aligners in the series.

To fabricate aligners or braces for a particular patient, the orthodontist first constructs a computer model of the patient's dentition. This model may be generated, for example, by taking an impression of the patient's dentition and then scanning the impression to digitize the impression for manipulation in a computer. Alternatively, the clinician may directly scan the patient's teeth with an intraoral scanner. The scanned data is then used to construct the computer model. In each case, the computer model includes one or more, preferably all teeth, in the patient's upper and/or lower jaws.

Once the computer model has been constructed, the orthodontist may manipulate individual ones of the model teeth to ultimately determine a target orientation of each tooth that provides a corrected dentition for each respective jaw and which addresses any malocclusion and ideally provides an aesthetic smile. Multiple computer models may be generated prior to treatment. Each model may include a unique orientation of one or more model tooth in the dentition and may successively and incrementally reposition one or more model teeth from an initial orientation to a target orientation according to a treatment plan.

The incremental repositioning of the model teeth is then reproduced in a series of fabricated molds of the teeth. An aligner is formed from each fabricated mold. Where there are multiple molds, a set of aligners is manufactured with each aligner being unique to one of the molds. When worn by a patient, each aligner imposes forces on the patient's teeth during orthodontic treatment. The patient's teeth may be moved incrementally from initial to target positions and orientations according to the treatment plan as determined by the computer models. In this way, treatment moves the patient's teeth in a series of stages from an initial orientation that generally corresponds to the initial orientation of the computer model to a final orientation that generally corresponds to the target orientation of the computer model.

Orthodontists often directly or indirectly prepare each stage of the treatment plan by providing specific instructions to the orthodontic appliance manufacturer. These instructions may include treatment goals for a patient. Those goals are a result of the orthodontist's examination of the patient's condition and are based on the orthodontist's experience and preferred treatment methods. Treatment goals may include specific instructions for individual tooth movement and may include a specific order of tooth movement by which the goals are to be obtained. The specific instructions are in the form of a text-based description prepared by the orthodontist and transmitted to the appliance manufacturer. The instructions are then interpreted by a technician at the appliance manufacturer. The technician is responsible for preparing the digital treatment plan based on the text-based description. Once prepared, the treatment plan may be transmitted to the orthodontist for final approval prior to manufacturing any orthodontic appliances. Once approved, the corresponding appliances designed to treat the patient's malocclusion are manufactured and shipped to the orthodontist or patient for use by the patient.

While successful, there are significant drawbacks to current treatment planning. Thus, improved systems, and methods are needed in orthodontic treatment planning of orthodontic appliances.

### SUMMARY

The present invention overcomes the shortcomings and drawbacks of methods and systems for treatment planning heretofore known for use in orthodontic treatment. While the invention will be described in connection with certain embodiments, it will be understood that the invention is not limited to those embodiments. On the contrary, the invention includes all alternatives, modifications and equivalents as may be included within the spirit and scope of the present invention. In one aspect of the invention, there is a system for orthodontic treatment planning for a patient. The system comprises a processor and memory coupled to the processor. The memory is configured to store computer-program instructions that, when executed by the processor cause the system to display a user interface on a display. The user interface is for a user to input a prescription for treatment of the patient. The user interface provides a plurality of commands for selection, wherein each command is a predetermined instruction based on orthodontic nomenclature for moving or modifying one or more of a patient's teeth. The computer-program instructions, when executed by the processor cause the system to receive a selected two or more of the plurality of commands into the prescription for treatment. The two or more selected commands are to be applied according to a predetermined protocol to the patient's teeth.

In one embodiment, the system further comprises a database coupled to the memory and accessible by the processor. The database is configured to receive the prescription for treatment of the patient and to contain a plurality of other prescriptions for treatment of other patients.

In one embodiment, the database is configured to receive a plurality of other prescriptions from a plurality of users of the system.

In one embodiment, the predetermined instruction is selected from a one-word instruction, a two-word instruction, and a three-word instruction or a combination thereof.

In one embodiment, the predetermined instruction is selected from the group consisting of a one-word instruction, a two-word instruction, and a three-word instruction.

In one embodiment, when executed by the processor, the computer-program instructions cause the system to limit a selected one of the plurality of commands to a single line in the prescription for treatment.

In one embodiment, when executed by the processor, the computer-program instructions cause the system to identify the two or more selected commands in the prescription for treatment for simultaneous application to the patient's teeth according to the predetermined protocol.

In one embodiment, when executed by the processor, the computer-program instructions cause the system to arrange the two or more selected commands in the prescription for treatment for sequential application to the patient's teeth according to the predetermined protocol.

In one embodiment, when executed by the processor, the computer-program instructions cause the system to arrange the two or more selected commands in the prescription for treatment for sequential application to the patient's teeth according to the predetermined protocol and identify at least one of the two or more selected commands for sequential application for simultaneous application with at least one other of the two or more selected commands to the patient's teeth according to the predetermined protocol.

In one embodiment, when executed by the processor, the computer-program instructions cause the system to prevent entry of text into the prescription for treatment that is not one of the selected commands.

In one embodiment, when executed by the processor, the computer-program instructions cause the system to display, in the user interface, a rectangular-shaped border encircling the predetermined instruction of each command.

In one embodiment, when executed by the processor, the computer-program instructions cause the system to stack two or more rectangular-shaped borders one above the next in the user interface.

In one embodiment, when executed by the processor, the computer-program instructions cause the system to save a combination of the stacked two or more selected commands as a user-defined command to the memory.

In one embodiment, when executed by the processor, the computer-program instructions cause the system to save the prescription for treatment in a machine-readable format.

In one embodiment, when executed by the processor, the computer-program instructions cause the system to generate a staging plan for review by an orthodontist.

In one embodiment, the system further comprises appliance manufacturing equipment configured to manufacture an appliance based on the prescription for treatment, wherein when executed by the processor, the computer-program instructions cause the system to transmit the prescription for treatment to the appliance manufacturing equipment and the appliance manufacturing equipment reads the prescription for treatment.

In one embodiment, there is a system for building an orthodontic treatment plan applicable to teeth of a patient. The system comprises a user interface for interfacing with a computer program. The computer program is configured to interact with a user through the user interface to display a representation of the teeth of the patient. The user selects one or more teeth from the representation of the teeth of the patient for treatment according to the orthodontic treatment plan. The computer program is configured to display a plurality of commands from a library of commands. The library of commands is predetermined. The displayed commands are predetermined instructions based on orthodontic nomenclature for moving the selected one or more of the teeth to a new position, and the user selects one or more of the plurality of commands for inclusion in the orthodontic treatment plan.

In one embodiment, the computer program is further configured to retrieve an initial position of the selected one or more teeth, determine a final position of the tooth subsequent to orthodontic treatment, and determine a staging plan for moving the tooth from the initial position to the final position based on the selected one or more of the plurality of commands in the orthodontic treatment plan.

In one embodiment, each predetermined command is selected from a one-word instruction, a two-word instruction, and a three-word instruction or a combination thereof.

According to another aspect of the invention, there is a computer-implemented method of creating an orthodontic treatment plan applicable to teeth of a patient. The method comprises receiving a digital model of a patient's teeth in a first arrangement and selecting one or more commands from a library of commands. The library of commands is predetermined. The method comprises placing the selected one or more commands into a prescription for orthodontic treatment of the patient, converting the prescription into machine-readable code for use by a processor of a computer, and creating a second digital model of the patient's teeth in a second arrangement different from the first arrangement based on orthodontic treatment according to the machine-readable code.

In one embodiment, the computer-implemented method further comprises determining a staging plan for moving or modifying the one or more of the patient's teeth from positions in the first model to positions in the second model based on the prescription for orthodontic treatment of the patient.

In one embodiment, the selected command is a one-word instruction, a two-word instruction, or a three-word instruction based on orthodontic nomenclature for moving or modifying one or more of the patient's teeth.

In another aspect, there is a computer-implemented method of building an orthodontic treatment plan applicable to teeth of a patient. The method comprises displaying a first digital model of the teeth of the patient to a user, receiving from the user a selection of one or more teeth of the first digital model, and displaying a plurality of commands from a library of commands. The library of commands is predetermined, and the displayed commands are instructions based on orthodontic nomenclature for manipulating the selected one or more of the teeth, each command manipulating the teeth in a distinct way from each other command. The method further comprises receiving from the user a selection of the one or more displayed commands. The selected commands make up the orthodontic treatment plan. The method further comprises creating a second digital model of the teeth of the patient by moving and/or modifying at least selected one or more teeth of the first digital model based on the orthodontic treatment plan.

In one embodiment, receiving a selection from the user of the one or more displayed commands includes the user selecting a one-word instruction, a two-word instruction, or a three-word instruction or a combination thereof from the library of commands, and creating the second digital model of the teeth includes applying the selected instruction to the selected one or more teeth.

In one embodiment, the computer-implemented method further comprises, after creating, displaying the created second digital model of the teeth of the patient to the user.

In one embodiment, the computer-implemented method further comprises, after creating, designing a mold using the second digital model, wherein the mold is usable in manufacturing an orthodontic treatment device.

In another aspect of the invention, there is a method of preparing a prescription for orthodontic treatment comprising selecting two or more commands from a library of commands. The library of commands is predetermined, and the selected command is a one-word instruction, a two-word instruction, or a three-word instruction based on orthodontic nomenclature for moving or modifying one or more of a patient's teeth. The method further comprises placing the selected two or more commands in a predetermined order of application into a prescription for orthodontic treatment of the patient.

In one embodiment, placing the selected commands includes placing a first command on a first line of the prescription and a second command on a second line of the prescription.

In one embodiment, the predetermined order of application is the first command first and the second command after the first command.

In one embodiment, placing includes grouping the first command and the second command.

In one embodiment, the predetermined order of application is simultaneous application of the first command and the second command.

In one embodiment, placing the selected commands includes placing a first command on a first line of the prescription and a second command on a second line of the prescription, and the method further comprises selecting a third command, placing the third command on a third line of the prescription, grouping the first command and the second command or the second command and the third command for simultaneous application to the patient's teeth according to the predetermined protocol, and arranging the first command or the third command for sequential application with the grouped commands to the patient's teeth according to the predetermined protocol.

In one embodiment, at least one of the two or more commands include a user-defined variable, and wherein following selecting two or more commands, and the method further comprises modifying the user-defined variable of the selected command.

### BRIEF DESCRIPTION OF THE DRAWINGS

The accompanying drawings, which are incorporated in and constitute a part of this specification, illustrate embodiments of the invention and together with the detailed description given below, serve to explain various aspects of the invention.

FIG. 1 is a schematic diagram illustrating one embodiment of an orthodontic appliance design and manufacturing system.

FIG. 2 is an exemplary flow according to one embodiment of a method of orthodontic treatment planning for a patient.

FIG. 3 is an exemplary user interface for use with a system of FIG. 1.

FIG. 4 is a schematic view of a system of FIG. 1 and according to one embodiment of the invention.

FIGS. 5A, 5B, 5C, 5D, and 5E are an exemplary user interface for use with a system of FIG. 1.

FIGS. 6, 7, and 8 are exemplary commands according to embodiments of the invention.

FIGS. 9, 10, 11, and 12 are exemplary strategies for combining the exemplary commands of FIGS. 6, 7, and 8.

FIG. 13 is a schematic diagram providing a protocol for execution of commands according to embodiments of the invention.

FIG. 14 is an orthodontic appliance in the form of an aligner according to one embodiment of the invention.

FIG. 15 is an orthodontic appliance in the form of an orthodontic bracket according to one embodiment of the invention.

FIG. 16A is an exemplary prescription with selected commands.

FIG. 16B is an exemplary treatment plan in the form of a staging plan in accordance with the prescription of FIG. 16A.

FIG. 16C is a plan view (top) of a patient's dental model for application of the prescription of FIG. 16A at stage 1 in FIG. 16B.

FIG. 16D is a plan view (top) of the model of FIG. 16C during application of the prescription of FIG. 16A and represents teeth position/orientations at stage 21 in FIG. 16B.

FIG. 16E is a plan view (top) of the model of FIG. 16C during application of the prescription of FIG. 16A and represents teeth position/orientations at stage 56 in FIG. 16B.

FIG. 17A is an exemplary prescription with selected commands.

FIG. 17B is an exemplary treatment plan in the form of a staging plan in accordance with the prescription of FIG. 17A.

FIG. 17C is a plan view (top) and an elevation view (bottom) of a patient's dental model for application of the prescription of FIG. 17A at stage 1 in FIG. 17B.

FIG. 17D is a plan view (top) and an elevation view (bottom) of the model of FIG. 17C during application of the prescription of FIG. 17A and represents teeth position/orientations at stage 21 in FIG. 17B.

FIG. 17E is a plan view (top) and an elevation view (bottom) of the model of FIG. 17C during application of the prescription of FIG. 17A and represents teeth position/orientations at stage 22 in FIG. 17B.

FIG. 17F is a plan view (top) and an elevation view (bottom) of the model of FIG. 17C during application of the prescription of FIG. 17A and represents teeth position/orientations at stage 23 in FIG. 17B.

FIG. 17G is a plan view (top) and an elevation view (bottom) of the model of FIG. 17C during application of the prescription of FIG. 17A, represents teeth position/orientations at stage 56 in FIG. 17B, and is an exemplary T2 model.

### DETAILED DESCRIPTION

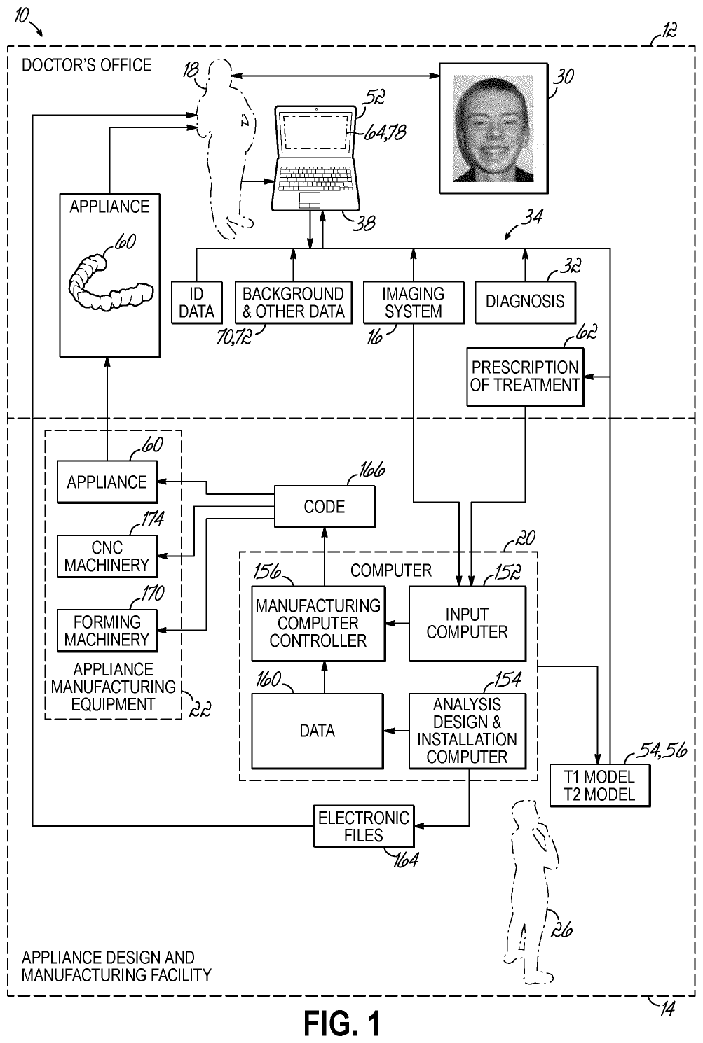

With reference to FIGS. 1, 2, and 3, there is an exemplary embodiment of an orthodontic appliance treatment and manufacturing system 10. Embodiments of the invention address problems identified with current orthodontic treatment planning. These problems include miscommunication between an orthodontist and an appliance manufacturer. As is described above, the orthodontist may prepare specific written instructions for patient treatment. Similar to a written prescription for pharmaceuticals, the written instructions for a patient's orthodontic treatment may be referred to as a prescription. Those written instructions may provide individualized treatment goals along with desirable tooth movement that are to be incorporated in a treatment plan for treatment of the patient with orthodontic appliances. The treatment plan may be prepared by a designer and/or an appliance manufacturer.

In a process of preparation of the treatment plan, the orthodontist's written instructions may be reviewed by a technician at the appliance manufacturer. Although embodiments are not limited to any relationship between the technician, who reviews the written instructions, and an appliance manufacturer, the technician may be employed by the appliance manufacturer. The technician may be responsible for incorporating the orthodontist's instructions into an initial treatment plan for treating the patient with appliances. This necessarily requires that the technician interpret the orthodontist's written instructions. Based on an interpretation, the technician then develops the initial treatment plan. It was discovered that the technician's interpretation of the orthodontist's written instructions frequently introduces unintentional deviations between the orthodontist's intended treatment and an initial treatment plan. As a result, the initial treatment plan is incorrect.

One exemplary cause of inaccuracy is that the technician's interpretation often involves translation between different languages. That is, the orthodontist may communicate a set of treatment goals for a particular patient in one language (i.e., their native language), and the technician at the appliance manufacturer may interpret those treatment goals based on a translation (i.e., to their native language, if different from the orthodontist's instructions) of the treatment goals. Another exemplary cause of inaccuracy is variation in the specific language the orthodontist uses to describe the intended treatment. For example, different orthodontists may refer to a similar treatment with different terminology. No matter how slight the differences, down to the order of the desirable tooth movement, the initial treatment plan prepared by the technician is incorrect. And, while corrections are made through back-and-forth communications between the orthodontist and the technician, that back-and-forth process wastes significant time and resources of both parties. Furthermore, oftentimes it takes time for a technician to learn and be proficient with an orthodontist's custom prescription preferences. Technicians can leave their employment, so once a technician understands the orthodontist's particular preferences, that understanding is lost if the technician leaves.

Embodiments of the invention, such as the system 10 shown in FIGS. 1-4, solves those problems and others. Misinterpretation of the orthodontist's instructions are eliminated by utilizing universal commands described herein. Through the universal commands, an orthodontist communicates with a technician and/or directly with a prescription system, which may include communication directly with a machine, with a standardized language having terms that are rooted in orthodontic treatment. The universal commands may be the only communication of a patient's prescription between the orthodontist and the technician and/or manufacturer. However, additional written notes may accompany the commands. The universal commands are instructions for orthodontic treatment with which the orthodontist may build a treatment prescription. The treatment prescription is ultimately used to create orthodontic appliances to move the patient's teeth according to the treatment prescribed in the prescription. The universal commands are one, two, or three-word instructions. The commands may be in verb or noun form. Each universal command has or is given a specific meaning in orthodontic treatment. That meaning is understandable by the orthodontist and by a technician and/or by a manufacturing facility/system to which the commands represent software code. With respect to automatic reading of the prescription by a manufacturing system, a technician's participation in the development of a treatment plan may be minimized or eliminated. In essence, the universal commands provide a standardized, common language between an orthodontist and a technician and/or between an orthodontist and a manufacturing facility/system, much like commands in software programming. The commands eliminate any need for interpretation by a technician.

Embodiments of the invention may additionally define a protocol for use of the universal commands. The protocol is predetermined and known by each of the orthodontist and the technician and/or has meaning for control of machines in the manufacturing system. Advantageously, with the protocol, an order of execution of single ones of the universal commands or individual ones of the universal commands in groups of universal commands is established. The universal commands and the protocol eliminate subjective interpretation of the meaning and an order of the orthodontist's instructions. Other advantages and benefits described herein also follow from the use of universal commands and corresponding protocol including, for example, automatic manufacturing of appliances via a computerized manufacturing system.

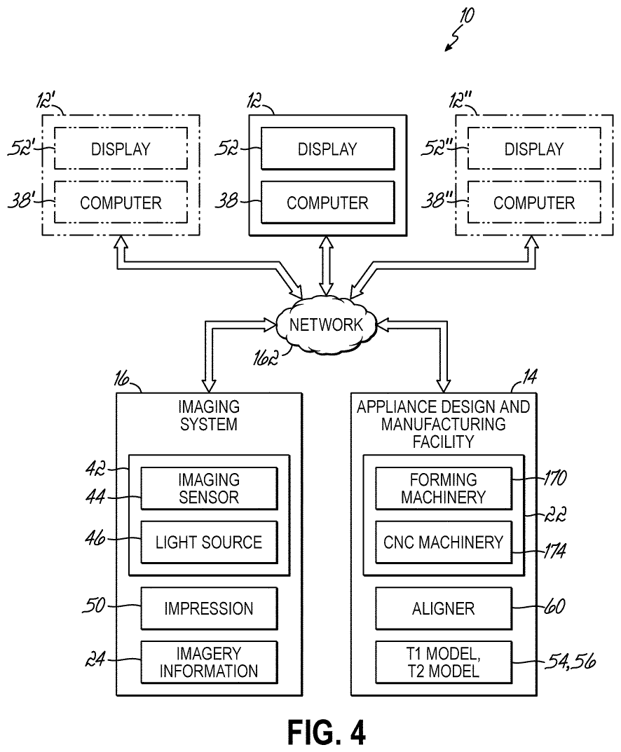

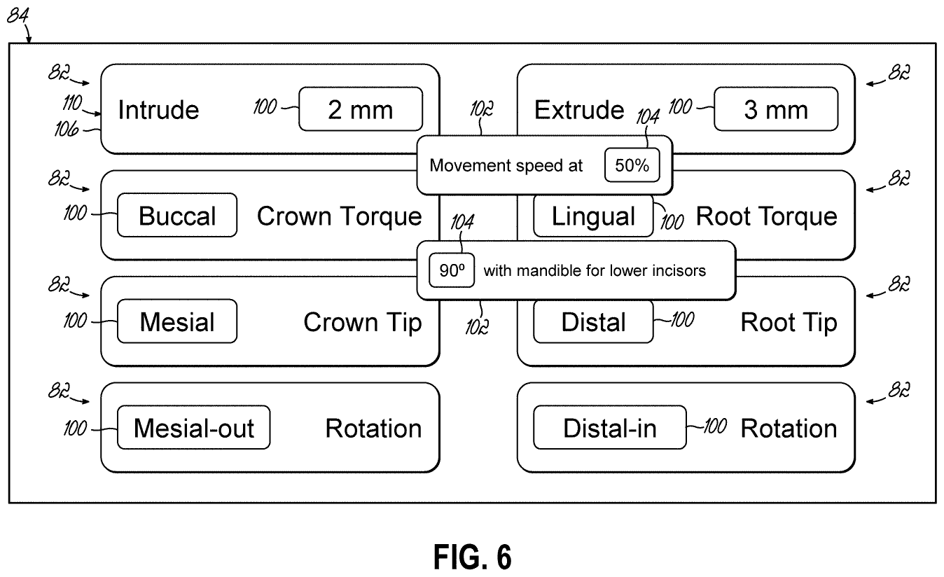

With reference to FIG. 1, in an exemplary embodiment and in general, the system 10 includes at least universal commands (shown, for example, in FIGS. 6-8 and described below) for communication of treatment information between an orthodontist's office 12 and an orthodontic appliance designing and manufacturing facility 14. With reference to FIGS. 1 and 4, the system 10 may be distributed among a plurality of locations, such as between the office 12 and facility 14 and multiple other offices 12′ and 12″. Embodiments of the invention are not, however, limited to any number of locations. For example, the system 10 may incorporate a single location though multiple locations are contemplated, as shown in FIG. 4 in which an imaging system 16 is at a separate office/building from separate orthodontist's offices 12, 12′, and 12″. In FIG. 1, the imaging system 16 is located at the orthodontist's office 12.

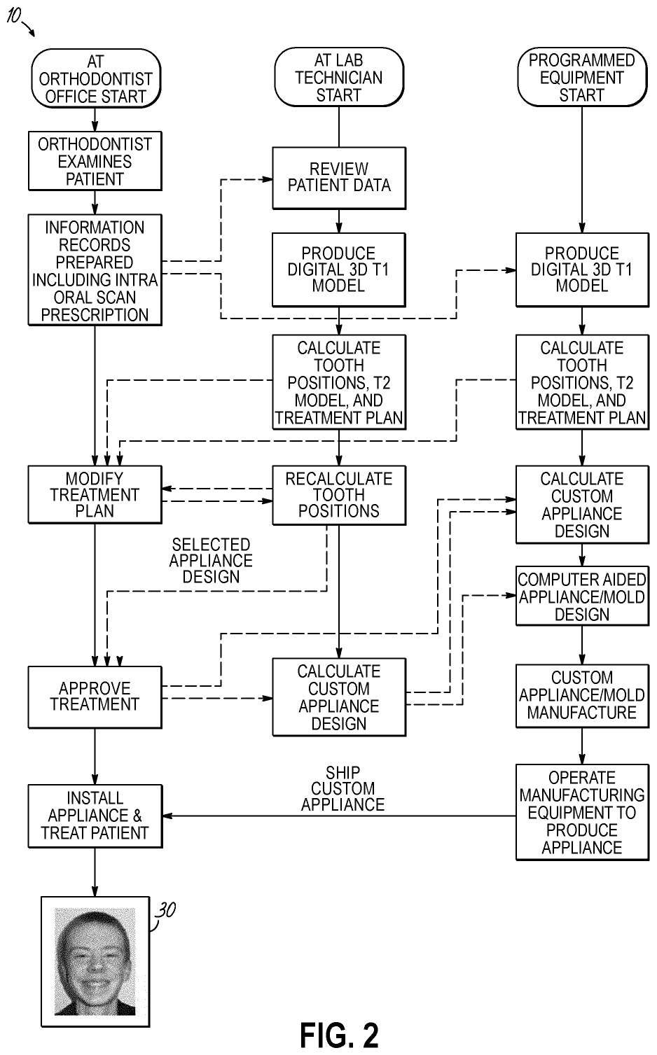

With reference to FIGS. 1 and 2, communication and decision making in the operation of the system 10 may be distributed among three paths, including (1) an orthodontist 18 located at the office 12, (2) data gathering and processing equipment 20 located at the facility 14 and/or at the office 12, and (3) the lab operator or technician 26 located at the facility 14 in which appliance manufacturing equipment 22 is housed. These three paths are represented by the three columns in FIG. 2. The decisions of the orthodontist 18 are illustrated at the left, decisions and steps performed by a technician 26 or by equipment 20 at the facility 14 are illustrated in the center, and the processes of the manufacturing equipment are illustrated at the right. The dashed lines in FIG. 2 represent interactions, including exchanges of universal commands, between the paths.

In one embodiment of preparing a treatment plan for a patient, the orthodontist 18 examines a patient 30 at the office 12 and makes a diagnosis 32 for orthodontic treatment. That diagnosis and treatment may be reduced in form to one of several data records 34 and entered in computer 38. The records 34 are generated as part of the case information necessary to determine the patient's condition, prescribe the appropriate treatment, and specify the orthodontic appliances to implement the prescribed treatment. The case information data records 34 include information identifying the patient 30, anatomical data from the patient 30, and other background information.

Referring to FIGS. 1 and 4, examination of the patient 30 by the orthodontist 18 involves the traditional application of the skill, knowledge, and expertise of the orthodontist 18, and results in the creation of a detailed anatomical record of the shape and initial malocclused locations of the teeth in the patient's mouth as well as the jaw structure of the patient 30. This detailed anatomical record may include information, such as imagery information 24 of the patient's jaws, from imaging system 16. The orthodontist 18 or someone under their direction may utilize the imaging system 16 to gather the imagery information 24 (FIG. 4) from the patient 30. For example, a clinician may insert at least a portion of a wand 42 into the patient's mouth. Using a light source 44 and an imaging sensor 46, the clinician may capture data of all or selected crowns of the patient's teeth. Data may include surface imagery and/or volumetric imagery (i.e., volumetric data acquired from, for example, cone beam computed tomography (CBCT) or similar device not shown) of one or more of the patient's teeth. Alternatively, surface imagery may be captured from an impression 50 of the patient's teeth. The imagery information 24 may be viewable on a display 52 coupled to computer 38 and may be transferred to the facility 14 at which virtual models, referred to as a T1 model 54 and a T2 model 56 may be prepared by the technician 26.

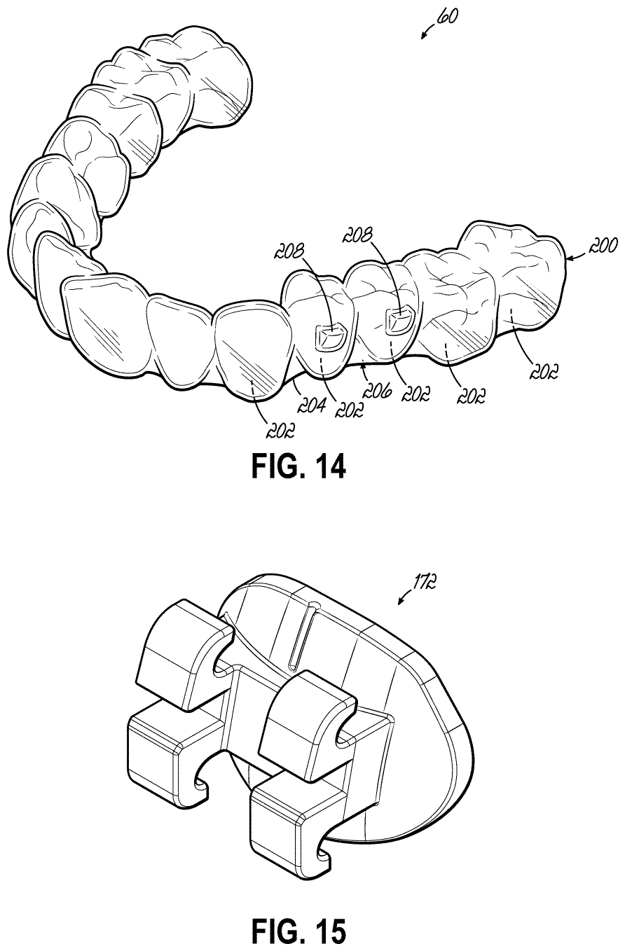

The orthodontist 18 determines the general type of orthodontic appliance (e.g., orthodontic aligner (FIG. 14) or orthodontic bracket (FIG. 15)) with which the patient 30 is to be treated, as well as certain parameters of such an appliance. As shown, in the system 10, the exemplary appliance is an aligner 60 (see, e.g., FIG. 14). To initiate production of the aligner 60 (typically as one of a series of aligners), the doctor's office 12 transmits the imagery information 24 to the facility 14 along with the other data records 34, including a prescription 62 in which the orthodontist 18 sets forth one or more universal commands for inclusion in an initial treatment plan for the patient 30.

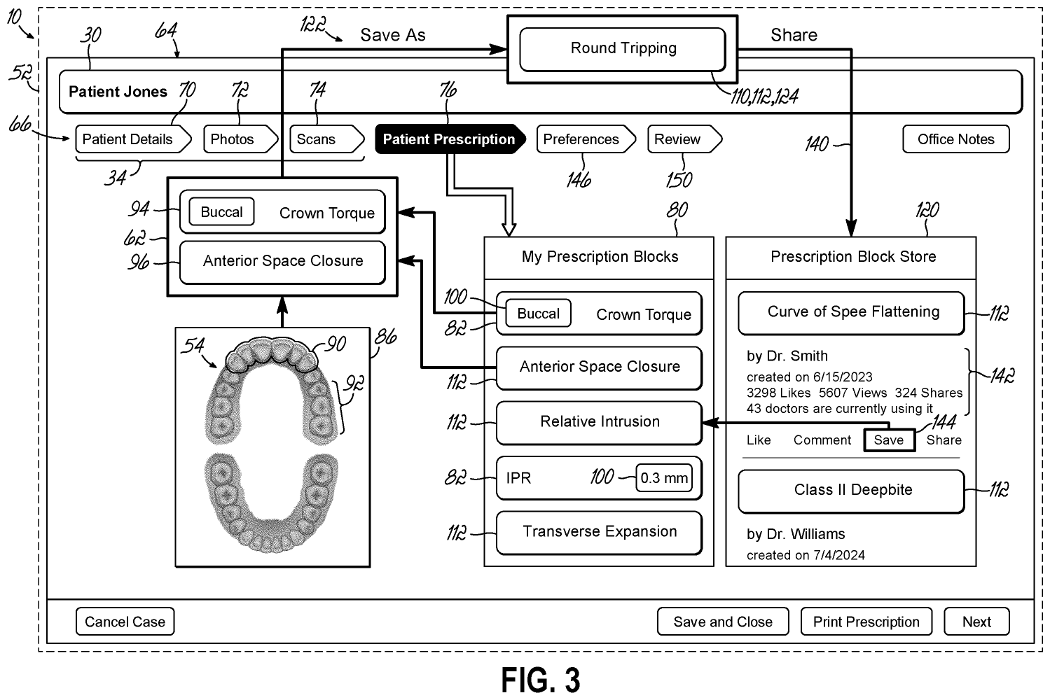

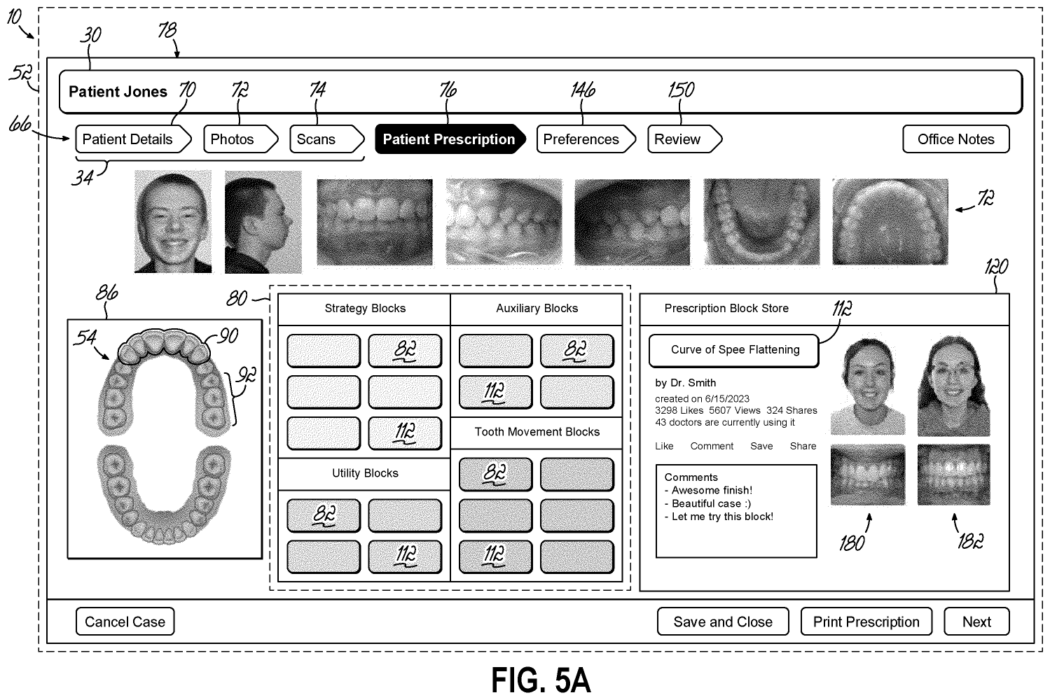

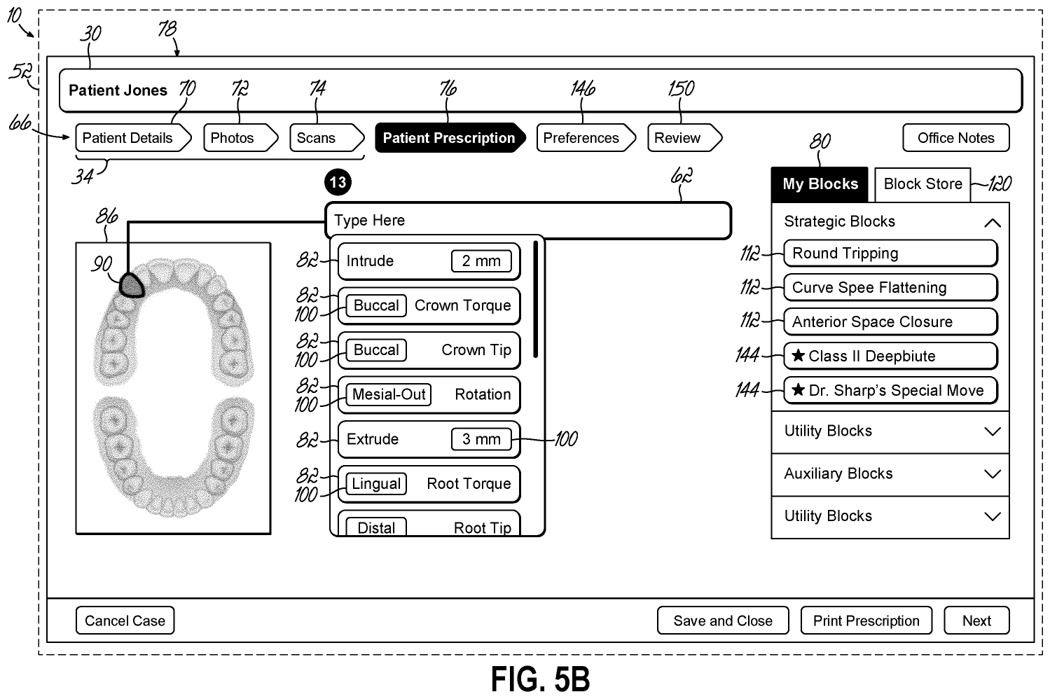

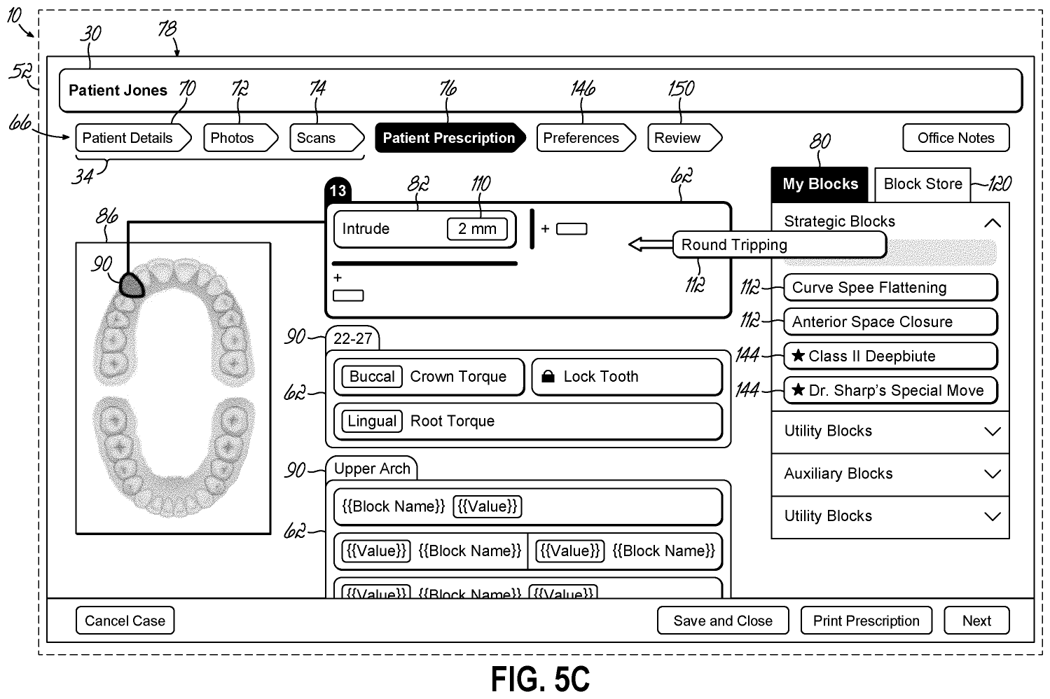

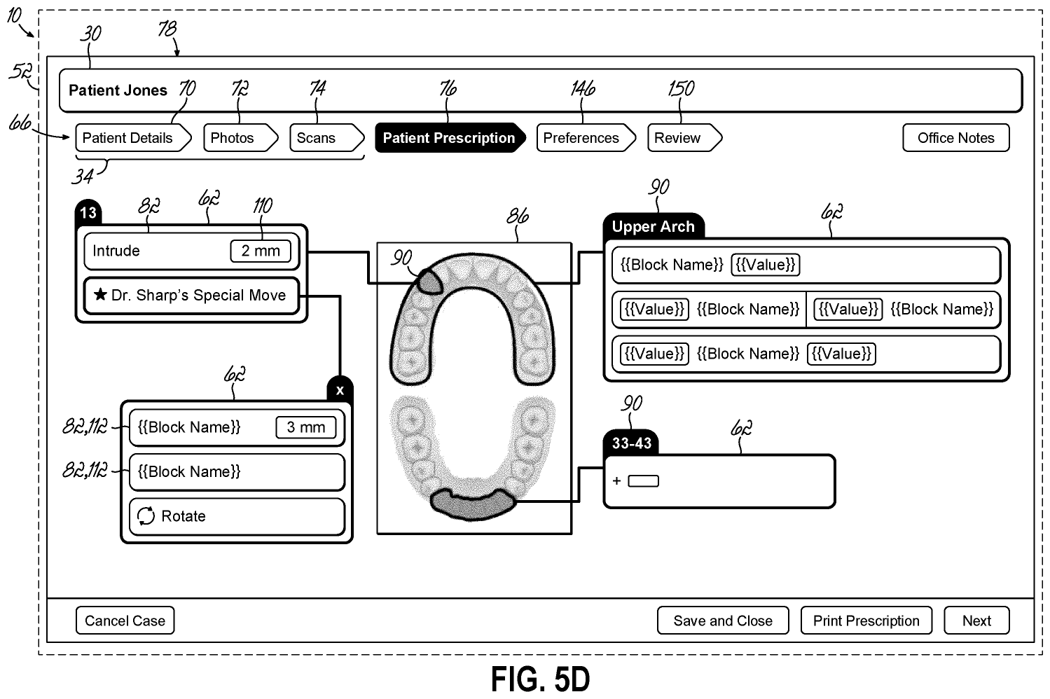

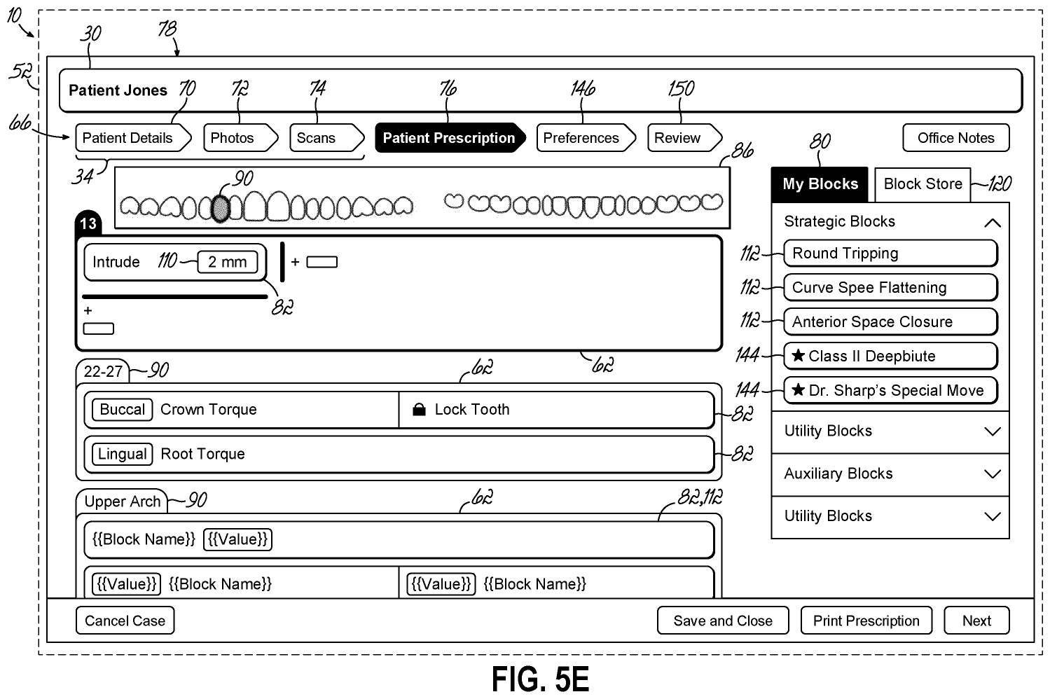

According to one embodiment of the invention, and with reference to FIGS. 1 and 3, the prescription 62 is generated via a user interface 64 on display 52. The orthodontist 18 enters the prescription 62 via interface 64 into computer 38 for transmission from the computer 38 to the manufacturing facility 14. The user interface 64 is designed to prompt the orthodontist 18 for information necessary to treat a particular patient. As shown in FIG. 3, in one embodiment, the user interface 64 includes a flowchart 66 by which the user interface 64 guides the orthodontist 18 in preparing data records 34 required to treat the patient 30. For example, the flowchart 66 guides preparation of information concerning each of “Patient Details” 70, “Photos” 72, “Scans” 74 (e.g., imagery information 24), and “Patient Prescription” 76. User interfaces according to embodiments of the invention are not limited that shown in FIG. 3. As example, other user interfaces 78 is described below with reference to FIGS. 5A-5E.

In FIG. 3, according to flowchart 66 at 76, the orthodontist 18 prepares the patient's prescription 62. To facilitate preparation, the user interface 64 depicts one or more commands 82 that the orthodontist 18 selects to build the prescription 62 for orthodontic treatment of the patient 30 (e.g., “Patient Jones”). The commands 82 are individually selectable from the prescription block 80 or from a library 84 (see FIGS. 6-8) for application in orthodontic treatment of the patient 30. As shown, the user interface 64 includes a prescription block 80 (i.e., “My Prescription Blocks”) that lists selectable universal commands 82 available to the orthodontist 18 to build the prescription 62. The prescription block 80 may also list selectable user-defined commands 112, described below, for building the prescription 62. The user-defined commands 112 may be used alone or in combination with commands 82 and/or in combination with other user defined commands 112. In the exemplary embodiment, the user interface 64 provides an image area 86 which may depict a patient image. By way of example only, and not limitation, the T1 model 54 may be shown in the image area 86.

In the interface 64, the commands 82, 112 displayed in the prescription block 80 may be linked to a specific tooth 90, a segment of teeth 92 in the T1 model 54, or the entirety of the T1 model 54 (e.g., the entire dentition, including both arches). For example, if the orthodontist selects the upper anterior teeth 90 on the T1 model 54, the commands 82, 112 available for selection in the prescription block 80 may differ from the commands 82, 112 available for selection in the prescription block 80 if the orthodontist 18 selects one of the more teeth 92 for treatment. In this way, the commands 82, 112 available for selection may be linked to and/or varied by the image displayed in the image area 86. Advantageously, this may make building the prescription 62 easier as only commands 82, 112 relevant to the image area 86, e.g., the selected teeth 90 or 92, are displayed in the prescription block 80.

The orthodontist 18 selects one or more commands 82, 112 for inclusion in the prescription 62. For example, in FIG. 3, the orthodontist 18 may select an anterior segment (canine to canine) 90 of an upper arch in the T1 model 54. With that selection, the “My Prescription Blcoks” 80 may list relevant commands for the anterior segment 90. The orthodontist 18 may select the “Crown Torque” command 94 and then “Anterior Space Closure” command 96 for inclusion in the prescription 62. Each of these commands 94, 96 is selected, such as dragging and dropping, from the prescription block 80 to the prescription 62. Alternatively, an input field may be made available and as the orthodontist 18 starts typing, a list of commands may appear in real-time for selection. This “Type Here” prescription 62 is shown by way of example in FIG. 5B with the “Type Here” prescription 62 field shown with reference to a selected tooth 90, which in this case is tooth “13” in the upper right quadrant of the patient's mouth according to the FDI teeth number system. The system 10 may prevent any non-command information from being added to the prescription 62. That is, no text, no punctuation, or information other than the commands 82, 112 may be placed/entered in the prescription 62. In this way, the prescription 62 may only include predefined instructions in the form of commands 82, 112. No undefined instructions may be incorporated in the prescription 62. Further, in one embodiment, the system 10 prevents illogical command combinations. These may be thought of as illegal with respect to actual orthodontic treatment, particularly if, when the commands are combined, they are known to not advance orthodontic treatment in a meaningful way. If the orthodontist 18 attempts to combine commands that are illegal in that sense, the system 10 may provide a warning or prevent entry of the command into the prescription.

As shown, in one embodiment, the orthodontist 18 arranges the commands 82, 112 in an intentional manner defined by a predetermined protocol. In FIG. 3, for example, the protocol may define an execution order from top to bottom in the prescription 62. That is, in the example, the prescription 62 provides that Crown Torque 94 is intended to be applied before Anterior Space Closure 96 as it relates to movement of the teeth 90. In this way, the orthodontist 18 arranges the commands 82, 112 in a hierarchy that predetermines which commands 82, 112 in the prescription 62 is to be executed first, second, third, and so forth. In one embodiment, the prescription 62 lists only one command per line. Thus, the prescription 62 is a listing of single commands with each command occupying its own line.

The prescription block 80 may be automatically populated from a library 84 (see FIGS. 6, 7, and 8) of universal commands 82 and available user-defined commands 112. In FIG. 3, fewer than all the commands 82 from the library 84, shown in FIGS. 6-8, are listed in the prescription block 80. In one embodiment, a subgroup of all the commands 82, 112 may be shown in the prescription block 80. That subgroup may be displayed based on the commands 82, 112 commonly utilized, those associated with a particular tooth or region of teeth 90 in the image area 86 (described above), or those recently used by the orthodontist 18. Thus, the relevant commands 82, 112 from the library 84 may be available for selection in the prescription block 80. However, the orthodontist 18 may directly access the library 84 at any time. A determination of the commands 82 and user-defined commands 112 in any subgroup for selection in the prescription block 80 is not particularly limited, though the selectable commands 82, 112 in the prescription block 80 should facilitate efficient assembly of the prescription 62.

In the exemplary library 84 shown in FIGS. 6, 7, and 8, a plurality of exemplary commands 82 is shown. The commands 82 are predetermined instructions, such as a one-word, two-word, or three-word instruction, typically in the form of, or including, a verb or a noun. While one-word, two-word, and three-word instructions are disclosed, embodiments of the invention are not limited thereto, unless otherwise specified. In the exemplary library 84, the words for each command are selected from orthodontic treatment nomenclature. The assigned meaning and thus function of each command is based on an industry definition. Additionally, or alternatively, a selected command may be defined internally to the system 10. That definition is provided to the orthodontist 18 and the technician 26 by the system 10. In that regard, embodiments of the invention are not limited to orthodontic treatment nomenclature. By way of example, the commands 82 may include terms and/or symbols unrelated to orthodontic treatment, if the meaning of those terms and symbols is communicated with the orthodontist 18 and the technician 26. In any respect, in the system 10, the meaning of each command 82 is known by each of the orthodontist 18 and the technician 26.

With reference to FIGS. 3, 6, 7, and 8, in one embodiment, many of the exemplary commands 82 include at least one user-defined variable 100. By way of example only and not limitation, a command may include one user-defined variable 100 and other command may include two user-defined variables 100. Further, in one embodiment, some commands lack user-defined variables. With reference to FIG. 6, for example, one command 82 may be to “Intrude” a particular tooth, which means to move the tooth into or toward the associated jawbone by a predetermined distance. This is an example of a one-word instruction. In this instance, the user-defined variable 100 is the predetermined distance and is shown as “2 mm.” If the orthodontist selects the command “Intrude” 82 from the prescription block 80 (or the library 84) to prepare the prescription 62, the system 10 via the interface 64 will allow the orthodontist 18 to set the distance 100 either from a drop-down list or via direct keyboard entry. Instead of being prompted by the system 10, the orthodontist 18 may select the user-defined variable 100 simply by clicking on it and then selecting the distance 100 from a list or entering the desired distance.

Selected ones of the commands 82 may also or alternatively include an additional option or parameter 102 by which predetermined information is selectable. For Intrude, for example, the orthodontist 18 may customize the Intrude command 82 with parameter 102 of “Movement speed at” and set a variable 104 to “50%.” Other parameters 102 and variables 104 are possible. Selection of any of the commands 82 described herein may be from the prescription block 80 and/or from the library 84 as described with reference to Intrude command. Further customization of the selected command, if applicable, may be in response to a prompt from the system 10 via the interface 64 or at the orthodontist's request via the user-defined variable 100 and/or the parameter 102.

Other exemplary commands 82 shown in FIG. 6 include “Crown Torque”, “Crown Tip”, “Rotation”, “Extrude”, “Root Torque”, “Root Tip”, and “Rotation.” The Crown Torque command means changing the torque of the tooth (a third order movement around an x-axis) while keeping the root apex stationary. This occurs when a force is applied and causes movement of the crown and the root of a tooth in a bucco-lingual direction. Crown Torque is an example of a two-word instruction. When Crown Torque is selected, the system 10 via the interface 64 may prompt the orthodontist, or the orthodontist may optionally select, a direction 100, such as “Buccal,” as shown, or “Lingual”, in which the selected crown is to be torqued, e.g., buccally or lingually, respectively. The Crown Tip command means changing the angulation of the tooth (a second order movement around a y-axis) while keeping the root apex stationary. This occurs when a force is applied to cause movement of the crown of a tooth in a mesio-distal direction without movement of the apex of the root. When Crown Tip is selected, the system 10 via the interface 64 will request a direction 100, such as “Mesial” as shown or “Distal”, in which the selected crown is to be tipped, e.g., mesially or distally, respectively. The Rotation command means changing the rotation of the tooth (a first order movement around a z-axis) with respect to either the mesial or distal contact points (i.e., hinge rotation), or with respect to the center of the crown. Stated another way, the tooth is turned about its long axis. When Rotation is selected, the system 10 via the interface 64 will request a rotational direction 100, such as “Mesial-out” as shown or “Mesial-in” or “Distal-in” or “Distal-out.” These are ideas of a hinge rotation in which one side of the tooth's position is maintained with the opposing side moving. The Extrude command means to move a tooth out of or away from the associated jawbone. When Extrude is selected, the system 10 via the interface 64 will request, or the orthodontist 18 can insert, a distance 100, such as “3 mm,” as shown, by which the selected tooth is to be extruded. The Root Torque command means changing the torque of the tooth (a third order movement around an x-axis) while keeping the tooth's crown center stationary. This occurs when a force is applied, and the root is moved buccally or lingually while the crown of the tooth maintains its position. When Root Torque is selected, the system 10 via the interface 64 request for the orthodontist 18 can insert a direction 100, such as “Lingual” or “Buccal,” in which the root is to be torqued, e.g., lingually or buccally, respectively. The Root Tip command means changing the angulation of teeth (a second order movement around a y-axis) while keeping the crown center stationary. This occurs when a force is applied to the root, and the root is moved mesially or distally while the crown maintains its position. When Root Tip is selected, the system 10 via the interface 64 will request a direction, such as “Distal” as shown or “Mesial,” in which the selected root is to be tipped, e.g., distally or mesially, respectively. The Rotation command is a duplicate above but in a distal direction.

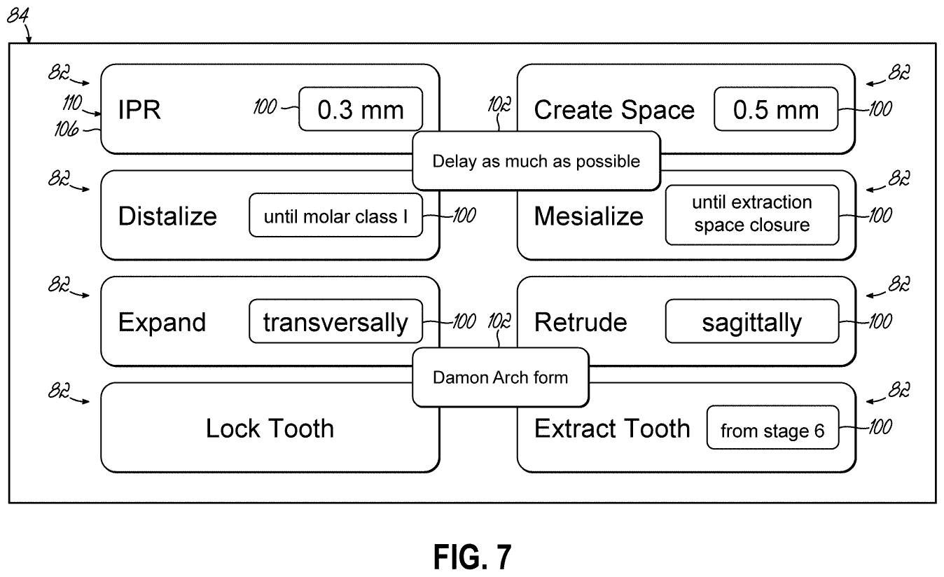

Other exemplary commands 82 are shown in FIG. 7 and include “IPR”, “Distalize”, “Expand”, “Lock Tooth”, “Create Space”, “Mesialize”, “Retrude”, and “Extract Tooth.” The IPR command means interproximal reduction, which refers to a procedure for removing a portion of the tooth surface. One specific purpose for interproximal reduction is to create more space between adjacent teeth. The user defined variable 100 is a dimension and is shown as “0.3 mm” and is the thickness of interproximal reduction required on the selected tooth. The orthodontist may optionally set timing 102 for when the IPR is to occur. In the example, timing is set to “Delay as much as possible.” The value of any parameter 102 for IPR may default to this value or other timing may be set by the orthodontist, for example, to “at Stage x” where x is the stage of treatment at which the IPR for the selected tooth is to occur. The Distalize command means moving the selected posterior tooth, such as a molar, distally. When Distalize is selected, the system 10 via the interface 64 will request a duration by which to move the selected tooth. By way of example, as is shown in FIG. 7, the user defined variable 100 is set to “until molar Class I,” which means to keep distalizing until a molar Class I relationship is established between the upper and lower teeth. In the proper position, the upper canine falls between the lower canine and the lower first premolar, and the mesio-buccal cusp of the maxillary first molar is aligned with the buccal groove of the mandibular first molar. The Expand command means to broaden the arch by moving the selected tooth outwardly relative to the narrow arch. Stated another way, the circumference of the dental arch is increased by moving the teeth buccally. This can be accomplished through tipping or translation, but it is usually accomplished with a combination of both tipping and translation. When Expand is selected, the system 10 via the interface 64 will request a direction 100, such as “transversally” or “radially.” The Expand command may be further modified by selection of an additional instruction, which in this case is a particular arch form. By way of example only, Expand transversally may be further customized by selection of a “Damon Arch form.” Thus, specific arch forms may be called out by the command. Other parameters 102 may include other arch forms, such as “Natural Arch form.” The Lock Tooth command means to maintain the location of a tooth during treatment. This command may not include a user-defined variable 100 and/or a parameter 102. The Create Space command means to create interproximal space mesial and/or distal to the tooth by moving the neighboring teeth away. this can be regarded as the conceptual opposite of IPR. When Create Space is selected, the system 10 via the interface 64 will request a dimension 100, such as “0.5 millimeters.” Other dimensions 100 include 0.2 mm, 0.3 mm, or 0.4 mm. A system default value may be 0.2 mm. The Mesialize command means moving the selected posterior tooth, such as a molar, mesially or toward the midline. When Mesialize is selected, the system 10 via the interface 64 will request a duration by which to move the selected tooth. By way of example, as is shown in FIG. 7, the user defined variable 100 is set to “until extraction space closure.” As an example, if a premolar is extracted and the orthodontist determines to mesialize the teeth distal to the extraction site of the premolar to close the space, mesialization will continue until the extraction space is closed completely under this option. The Retrude command means to move a crown of an anterior tooth lingually. When Retrude is selected, the system 10 via the interface 64 will request a direction 100, such as “sagittally.” The Extract command means that the selected tooth is to be extracted. When Extract is selected, the system 10 via the interface 64 will request a time 100, such as “from stage 6,” which means that the selected tooth is to be extracted at the specified stage. However, extraction of a tooth may be at another stage of treatment, such as at stage 1, stage 2, stage 3, stage 4, or stage 5.

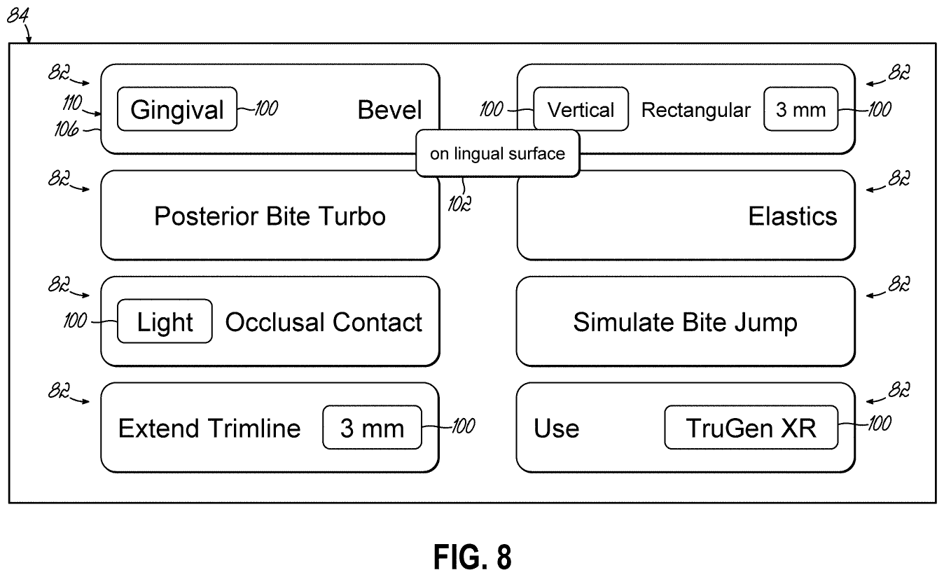

Additional exemplary commands 82 are shown in FIG. 8 and include “Bevel”, “Posterior Bite Turbo”, “Occlusal Contact”, “Extend Trimline”, “Rectangular”, “Simulate Bite Jump”, and “Use.” The Bevel command means adding a bevel attachment to the tooth surface. The user defined variable 100 for Bevel is a direction and is shown as “Gingival” meaning that the bevel surface of the attachment points toward the gingiva. That is, a thinner part of the attachment will be towards the gingiva direction. Other possible directions include “Occlusal,” “Mesial,” and “Distal.” Other exemplary user defined variables 100 include a size of the bevel attachment in millimeters. The Posterior Bite Turbo command means adding a bite turbo at a specified location. Posterior Bite Turbo may not include a user defined variable 100 and/or a parameter 102. The Occlusal Contact command means teeth on opposing jaws will have a contact point between each other on their occlusal surface. Occlusal Contact can be obtained by moving the tooth up or down in the jaw, for example. When Occlusal Contact is selected, the system 10 via the interface 64 will request a type of contact for the selected tooth. By way of example, as is shown in FIG. 8, the user defined variable 100 is set to “Light,” which means a light amount of biting contact, which depends on the virtual overlap of teeth. Other exemplary variables 100 include “Heavy.” Instead of qualities, these contacts may be quantitative, such as “0.1 mm,” “0.2 mm,” and “0.3 mm.” The Extend Trimline command determines where the aligner will be trimmed and can be used to raise the trimline (occlusally) and/or lower the trimline (toward the gingiva), for example, to cover exposed root surface in areas of tissue loss. When Extend Trimline is selected, the system 10 via the interface 64 will request a dimension 100, such as “3 mm.” Other user defined dimensions 100 may be set to customize the Extend Trimline command. The Rectangular command means adding a rectangular attachment to the tooth surface. Rectangular may be further modified by one or two of the user defined variables 100. As shown, customization is possible by setting a dimension for Rectangular, such as “3 mm,” which is a size of the rectangular attachment in millimeters. A second user defined variable 104 is a direction. As shown, Rectangular is customized by “Vertical.” Collectively, in the example, Rectangular is defined as a 3 mm rectangular attachment placed vertically on the tooth surface. The Elastics command means that the orthodontist intends to couple an elastic to the tooth. The Simulate Bite Jump command means to simulate a bite jump, which is a virtual simulation of jaw movement caused by the use of elastics, for example, showing the anteroposterior correction after the use of elastics or caused by surgery. This command may not include a user defined variable 100 and/or a parameter 102 and is an example of a three-word instruction. The Use command means to use a specific material or manufacture's product in the manufacture of an appliance. When Use is selected, the system 10 via the interface 64 will request a material type 100, such as “TruGen XR,” which is a registered trademark of Ormco Corporation. Alternative material types 100 include “TruGen.”

With reference to FIGS. 3, 6, 7, and 8, in one embodiment, the commands 82, 112 are each shown in an enclosed border 106. In the exemplary embodiment, each border 106 defines a rectangular-shaped block 110. As such, commands 82 may be referred to as “basic blocks” herein. The user-defined commands 112 may also be bordered and have a rectangular-shape. As such, these may be referred to as “user-defined blocks” herein. Each of the blocks 110 contains one command 82 and each of the blocks 110 is of similar size and shape. This uniformity in the size and shape of the blocks 110 is advantageous. The block-based appearance visually represents the commands 82 as building blocks. Thus, in one embodiment, the system 10 provides an environment in which the orthodontist 18 may visually build the prescription 62 from blocks, i.e., a block-based command structure. When complete, the prescription 62 structurally appears as an arrangement of blocks on individual lines of the prescription 62 and so is easily understandable. While rectangular-shaped blocks are shown, embodiments of the invention are not limited to the enclosed border 106 or any shape of the enclosed border 106 (i.e., square, triangular, round, etc.). As an example, the border 106 may be designed with internal logic or external shapes to visually indicate which commands 82 may be usable together and which commands 82 cannot be used together. This may prevent commands 82 not usable together, as described above, from being placed in a prescription together. In that regard, the border 106 may define a puzzle-like piece and so visually indicate any one or two command 82 that fit together. Conversely, the puzzle-like pieces also indicate when any two commands do not fit together. In addition, although not shown, the enclosed border 106 may be filled with a color so that the commands 82, 112 are color coded. Color coding may be advantageous for grouping of the commands 82, 112 according to their function, according to application to specific groups of teeth, or for other reasons, such as to distinguish basic blocks 82 from user-defined blocks 112.

With reference to FIGS. 3, 9, 10, 11, and 12, in one embodiment, the system 10 provides for building the user-defined commands 112. In this way, the orthodontist 18 may construct their own commands. These can be saved in the system 10. The user-defined command, which may be referred to as “Strategy Blocks,” are based on one or a combination of commands 82, 112 from the library 84. As the name suggests, the orthodontist 18 may prepare unique treatment strategies via the user-defined commands 112. Once initially built, the user-defined commands 112 may then be saved in the system 10 for later use by the orthodontist 18 or by other orthodontists in other offices 12′ and 12″ via, for example, network 162 (see FIG. 4), as is generally indicated by the Prescription Block Store 120 in FIG. 3 and described below.

By way of example, in FIG. 3, the orthodontist 18 may build the prescription 62 with a combination of Crown Torque 94 (i.e., a basic block 82) and then Anterior Space Closure 96 (i.e., a strategy or user-defined block 112). This combination of basic and user-defined commands 82, 112 may then be saved as is depicted at 122 to an orthodontist-generated name, in this case, “Round Tripping” 124. Thus, the orthodontist 18 may construct additional user-defined commands 112 from combinations of commands 82 and/or previously defined user-defined commands 112. As shown, the user-defined commands 112 may be depicted as a rectangular-shaped block 110 and so have visually similar appearance to the commands 82, which may be referred to as “basic blocks” or “system level codes” herein. Although not shown, the user-defined commands 112 may be given a different fill color to visually distinguish them from the commands 82. As an example, the basic blocks 82 may include a gray-colored fill whereas the user-defined commands 112 may include a blue-colored fill. However, any color coding is not limiting to embodiments of the invention.

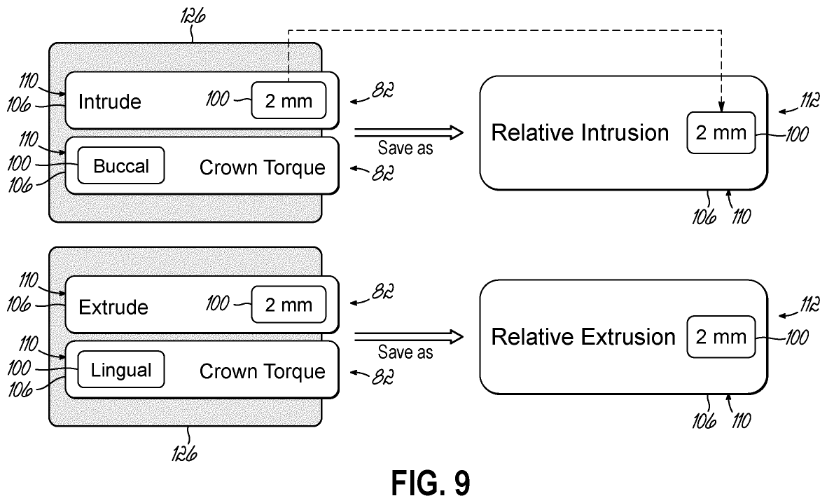

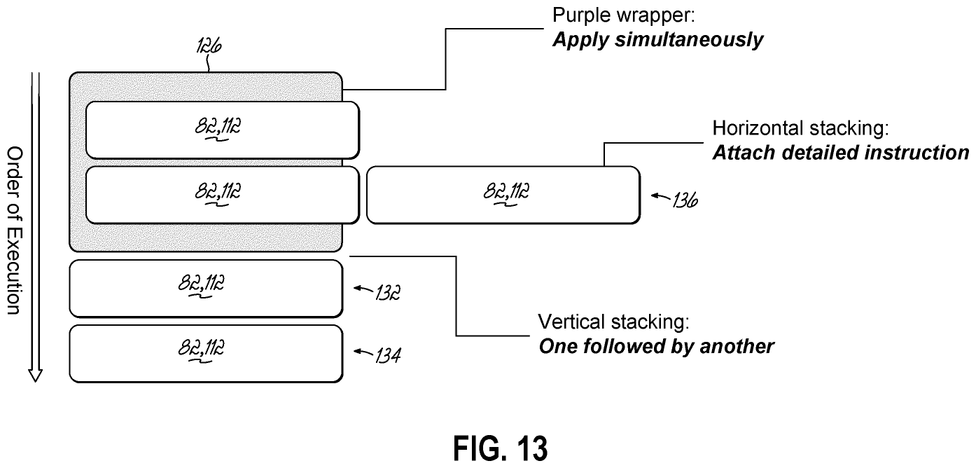

Other exemplary user-defined commands 112 are shown in FIGS. 9-12. With reference to FIG. 9, two different user-defined command codes 112 are shown. One is named “Relative Intrusion.” It is a combination of two basic blocks including the Intrude command 82 and the Crown Torque command 82. In addition, a relationship between the two commands 82 is visually indicated by a shaded block 126. In one embodiment, and with reference to FIG. 13, the shaded block 126 may be referred to as a “wrapper” which visually indicates a protocol as it relates to the commands 82, 112 that are enclosed by the block 126. In the case of FIG. 9, which schematically depicts only a part of a larger user inference, such as that shown in FIG. 3, the wrapper 126 determines an order of application between Intrude and Crown Torque as to selected teeth, segments of an arch, or the entirety of one or both arches. By way of example only, the protocol indicated by wrapper 126 is to simultaneously apply each of the Intrude and Crown Torque commands to the selected tooth. Thus, the wrapper 126 supersedes a sequential order of application defined by a protocol without the wrapper 126. For example, and with reference to FIG. 13, below the wrapper 126 there are two commands 82, 112. In the absence of wrapper 126, an exemplary protocol may be to apply the command 132 before the command 134. Overall, in FIG. 13 then, and exemplary order of execution is to first apply the commands 82, 112 in the wrapper 126 simultaneously, then the command 132, and lastly the command 134. Advantageously, the system 10 provides a visually structured, predefined language for preparing prescriptions 62. Moreover, the protocol, as a general rule, defines a single command per line. The protocol also defines exceptions to that general rule.

In that regard, commands may be arranged to be simultaneously applied. In one embodiment, the wrapper 126 specifies which commands are to be applied simultaneously. In FIG. 13, each command 82, 112 occupies a single line of code and so each command 82, 112 is sequentially applied according to the general one-command-per-line rule. By way of additional example, the protocol may include other variations of the general rule. One is horizontal stacking (e.g., side-by-side commands) in which the commands are executed “with” the next command to the right. For example, where the command line is “Command A Command B,” Command A is executed with the details of Command B. More specifically, for example, a “Lingual Root Torque” command with a “Gingival Bevel Attachment” on the “Lingual of the Crown Surface” means lingual root torque movement done with a gingival bevel attachment on the lingual crown surface of the tooth. Horizontal stacking is a higher priority than wrapping in the protocol, that is, it is applied first.

Referring, once again, to FIG. 9, the user-defined commands 112 “Relative Intrusion” is a combination of two system commands 82, that is, “Intrude” for “2 mm” and “Crown Torque” in a “Buccal” direction. Per the protocol defined by the wrapper 126, each of Intrude and Crown Torque are to be simultaneously applied. As shown, the user-defined commands 112 may retain one or more of the user defined variables 100 of the commands 82. In the exemplary embodiment shown, Relative Intrusion 112 retains the user defined variable distance 100. Once saved in the system 10, the orthodontist 18 or another orthodontist in offices 12′ and/or 12″ may select Relative Intrusion 112 and then modify the distance 100 to a value different from the 2 mm shown. Similarly, a “Relative Extrusion” user-defined command 112 is a combination of two system commands 82. The orthodontist 18 may combine them with the wrapper 126 such that Relative Extrusion and Crown Torque in a lingual direction are applied simultaneously. This combination of two commands 82 may then be saved is the user-defined Relative Extrusion command 112, which may retain the variable distance 100. Once saved in the system 10, the orthodontist 18 or another orthodontist in offices 12′ and/or 12″ may select Relative Extrusion 112 from the “My Prescription Blocks 80 of the user interface 64 (FIG. 3) and then modify the distance 100 to a value different from the 2 mm shown for inclusion in the prescription 62.

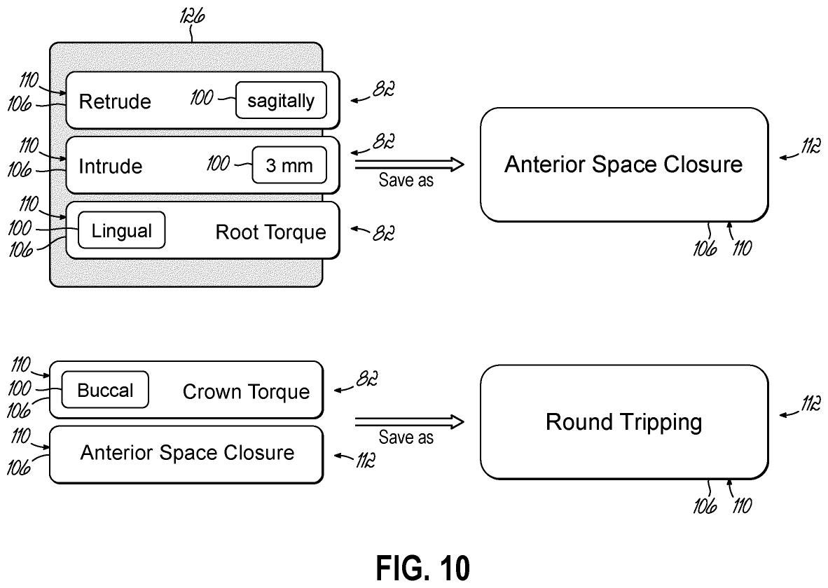

Additional exemplary embodiments of user-defined commands 112 are shown in FIG. 10. As shown, an “Anterior Space Closure” user-defined command 112 is a combination of three commands 82, which are to be simultaneously applied. That is, each of Retrude, Intrude, and Root Torque shown on the left side of the figure are to be simultaneously applied according to a protocol identified by the wrapper 126. In this example, each of the user defined variables 100 found in each of the commands 82 may not be assessable directly through the user-defined command 112. Once saved in the system 10, Anterior Space Closure 112 may appear in the “My Prescription Blocks” 80 (FIG. 3) of the user interface 64 and/or in the library 84. The system 10 permits the orthodontist 18 and/or another orthodontist to select it from the “My Prescription Blocks” 80 of the user interface 64 for inclusion in the prescription 62.

In the other example shown in FIG. 10, the user-defined command “Round Tripping” 112 is a combination of one command 82 and a previously constructed user-defined command 112. In the example, Crown Torque 82 with a user defined variable 100 of “Buccal” is combined with the Anterior Space Closure 112, described above and also shown in FIG. 10. According to the protocol, Crown Torque 82 is to be first applied and then Anterior Space Closure 112 is applied. The Anterior Space Closure 112 is a combination of simultaneously applied Retrude, Intrude, and Root Torque with their associated user defined variables 100 as shown in FIG. 10. Advantageously, the user-defined commands 112 permit the orthodontist 18 to construct groups of one or more commands 82 and one or more user-defined commands 112 together with or without a wrapper 126. The orthodontist 18 may save those unique combinations in the system 10 for later use or for later combination with other commands 82 and/or user-defined commands 112. This ultimately saves the orthodontist 18 time for building prescriptions that may require similar prescriptions 62 or include orthodontist preferred tooth movement and/or tooth movement sequences.

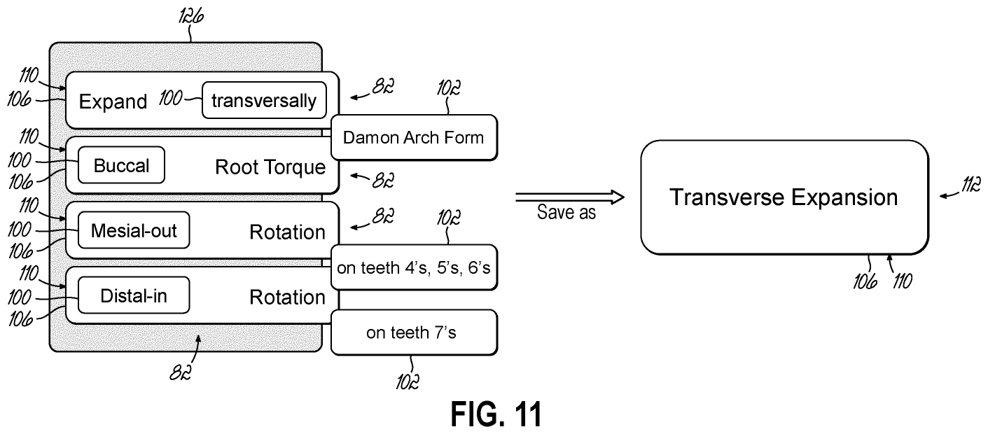

Another exemplary user-defined command 112 is shown in FIG. 11. In this example, an orthodontist-named command, i.e., “Transverse Expansion,” is constructed of four commands 82 which are simultaneously applied according to the wrapper 126. That is, each of Expand (transversally), Root Torque (buccal), Rotation (mesial-out), and Rotation (distal-in) are simultaneously applied. As is provided in the parameter 102, Expand (transversally) requires that “Damon Arch Form” be applied with Rotation (mesial-out) being applied “on teeth 4's, 5's, and 6's” and Rotation (distal-in) being applied “on teeth 7's,” all at the same time. As an example, on the upper arch “on teeth 4's, 5's, and 6's” and “on teeth 7's,” is with reference to the FDI (ISO 3950) system and refers to teeth 14, 24, 15, 25, 16, and 26 and 17 and 27, respectively. Once saved in the system 10, Transverse Expansion 112 may appear in the “My Prescription Blocks” 80 (FIG. 3) of the user interface 64 and/or in the library 84. The orthodontist 18 and/or another orthodontist at another office 12′ and/or 12″ may select it from the “My Prescription Blocks” 80 of the user interface 64 for inclusion in the prescription 62.

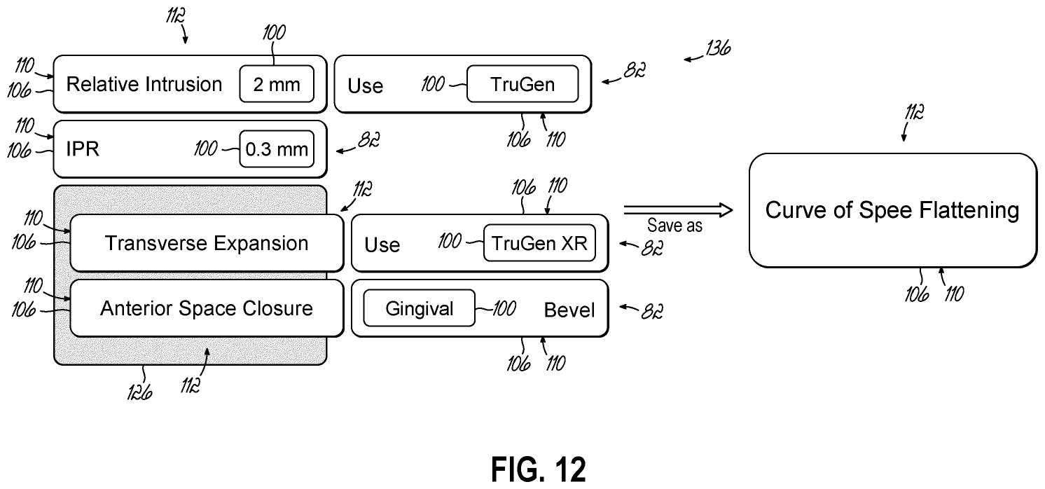

Another exemplary user-defined command 112 is shown in FIG. 12. As shown, the protocol further includes horizontal stacking of commands 82, 112 as indicated at 136 in FIG. 12. In this example, an orthodontist-named command code, i.e., “Curve of Spee Flattening,” is constructed of seven commands 82 and/or 112. Each of the initial three commands 82, 112 from the top is be applied first, of which two are applied in series. The remaining four are then simultaneously. Specifically, a combination of one user-defined command 112 (e.g., Relative Intrusion of FIG. 9 with Use command of TruGen) and one basic command 82 (e.g., IPR of 0.3 mm) are applied in series. Subsequently, two user-defined commands 112 are simultaneously applied. As shown, Transverse Expansion from FIG. 11 with Use command of TruGen XR and Anterior Space Closure from FIG. 10 with Bevel are wrapped together according to the wrapper 126. Once saved in the system 10, Curve of Spee Flattening 112 appears in the “My Prescription Blocks” 80 (FIG. 3) of the user interface 64 and/or in the library 84. The orthodontist 18 may select it from the “My Prescription Blocks” 80 of the user interface 64 and/or another orthodontist at another office 12′ and/or 12″ may select it from the store 120 of the user interface 64 for inclusion in the prescription 62. Advantageously, the system 10 permits customization of commands so that the orthodontist 18 may design and build treatment strategies, for example, by combining “Strategy Blocks,” “Auxiliary Blocks,” “Utility Blocks,” and “Tooth Movement Blocks,” for example, shown in FIG. 5A. Once built and saved, those orthodontist-created treatment strategies may be reused and/or customized for other patient prescriptions without reconstructing the entirety of the prescription.

Referring to FIG. 3, any or all user-defined commands 112 described above or others may be shared via the system 10 with other orthodontists at 140 to store 120. The store 120 is accessible by other orthodontists using the system 10. In FIG. 4, the store 120 having common commands may be shared on computers 38, 38′, and 38″ and so may be displayed in the store of interfaces 64 on each of displays 52, 52′, and 52″. As shown in FIG. 3, Dr. Smith shares the user-defined command “Curve of Spee Flattening” 112 and Dr. Williams shares the user-defined command “Class II Deepbite” 112 with other orthodontists who have access to the store 120. As an example, Dr. Smith can review Dr. Williams' Class II Deepbite command and comment on, like, and share it at 144. Similarly, Dr. Williams can review Dr. Smith's Curve of Spee Flattening command and comment on it, like it, and share it.

As is also shown, the store 120 may provide treatment efficacy information concerning the shared user-defined commands 112. In that regard, the store 120 may provide a forum by which multiple offices 12, 12′, 12″ may be connected for sharing user-defined commands 112 and prescriptions 62 between a plurality of orthodontists. For example, for Dr. Smith's shared command 112, at 142, the store 120 indicates a “created on” date and provides additional information regarding the “likes”, “views”, and “shares” that may provide some peer-reviewed indication of the efficacy of Dr. Smith's shared command 112. If the orthodontist 18 desires to incorporate Dr. Smith's “Curve of Spee Flattening,” the orthodontist 18 may save it at 144 to their prescription block 80 for use in their practices. The system 10 may record in a database each user-defined command 112 and/or prescription 62 across all users of the system 10 and the number of times that command has been used in a treatment plan. Thus, the system 10 may record information sufficient to develop quantitative information on command usage, user-defined command development, and prescriptions. In this way, for example, the system 10 may track the most-used commands, the most “likes,” “views,” and “shares” of user-defined commands and the most “likes,” “views,” and “shares” of prescriptions so that it is possible to determine which orthodontists are most influential with regard to one or more of the number of likes, views, and/or shares within the system 10. Additionally, prescriptions saved can be used to define clinical preferences for a specific user. Those clinical preferences may be general guidelines/rules to be universally applied to different patients for the specific user, and perhaps others who may adopt those clinical preferences.

Once the orthodontist 18 has constructed the prescription 62 for the patient 30, the flowchart 66 guides the orthodontist 18 through each of preferences 146, and review 150 and prior to submission of the prescription 62 to the facility 14.

A similar prescription may be built via any single one of the exemplary user interfaces 78 of FIGS. 5A, 5B, 5C, 5D, and 5E in which alternative information may be provided to and arranged for the orthodontist 18. As shown in each figure, the user interface 78 is shown on display 52 available to the orthodontist 18. With reference to FIG. 5A, the user interface 78 differs from the interface 64 of FIG. 3 in that the prescription block 80 may be subdivided into various categories of commands. For example, the prescription block 80 may be divided into 4 categories of commands referred to as blocks with “Strategy Blocks”, “Auxiliary Blocks”, “Utility Blocks”, and “Tooth Movement Blocks.” By subdividing the prescription block 80 into command categories, the user interface 78 may ease identification and selection of the commands 82 available in the library 84. As is indicated then each of the categories, both system commands 82, i.e., basic blocks, and user-defined commands 112 may be appear in each of the command categories. The user interface 78 also includes a series of photographs 72 in addition to the T1 model 54 in image area 86. The image area 86 may, instead of the T1 model 54, depict a representation of the teeth of the patient. That representation may be generic to all patients or include sufficient information by which the orthodontist 18 may identify the patient by their teeth, such as a digital photo of the patient's teeth. And, in the store 120, before and after photographs 180, 182, respectively, of a patient may be shared as evidence of an orthodontist's success using a particular prescription on that patient. Variations of the user interface 78 are shown in FIGS. 5B-5E in which the prescription 62 may be built from blocks 82, 112 from a library 84, the clinician's blocks 80, and/or from the block store 120. Selected blocks may be associated with selected teeth 90 (e.g., tooth 6, teeth 22-27, “Upper Arch” in FIGS. 5B, 5C, 5D, and 5E).

Referring to FIG. 1, in one embodiment, after submission of the prescription 62, the technician 26 may review the data records 34, including the prescription 62 and imagery information 24. This data may be received into the input computer 152 specifically dedicated to the design of the appliance 60. The technician 26 may also add input to or control operation of appliance manufacturing equipment 22 controlled by computer 154, such as machine controller 156 and/or to manufacture the appliance 60. Where the inputting, design, and manufacture are performed at the appliance facility 14, the computers 152, 154, and 156 may be the same computer or separate computers or controllers that are linked to each other or otherwise exchange data 160.

The imagery information 24 received from imaging system 16 may be reviewed by the technician 26 and entered into the input computer 152. The technician 26 via computer 152 may manipulate the imagery information 24 to provide the T1 model 54 and the T2 model 56. The technician 26 may also construct a treatment plan. The treatment plan may include a staging chart and may be a precise prediction of the prescribed treatment based on the T1 model 54 and the T2 model 56 in conjunction with the data records 34, including the prescription 62, from the orthodontist 18. The proposed treatment, the T1 model 54, the T2 model 56, and the treatment plan are communicated to the orthodontist 18 through a network 162 (FIG. 4) between facility 14 and the office 12. The orthodontist 18 may modify the initial treatment plan in response to which the design computer 154 recalculates the final treatment positions of the teeth and generates display data for further review, revision, or approval by the orthodontist 18. Algorithms may also be used to determine the treatment plan from the commands 82, 112 of the prescription 62 with little or no technician interaction. That is, the prescription 62 in the form of commands 82 arranged per the protocol may be machine-readable code and so dispense with the need for a technician. Alternatively, the prescription 62 may be converted to machine-readable code by the technician. In this case, the input computer 152 may automatically construct a treatment plan based on at least the prescription 62 and the T1 model 54. That information may be automatically transferred to the orthodontist for approval review.

Once the tooth treatment positions are approved by the orthodontist 18, the computer 156 automatically designs one or more appliances 60, 172 or molds for manufacturing appliances under the supervision of the technician 26. As a digital design is produced, the design information, which includes three-dimensional design display and numerical design data, may be provided over the network 162 to the computer 38 for interactive adjustment and ultimately approval by the orthodontist 18.

When the design has been approved by the orthodontist 18, the analysis and design computer 154 may produce archive files 164 that are written with all of the relevant information of the analysis and the history and prescribed treatment of the patient 30. Calculated information for the patient 30 may be stored in a patient data file. From the calculations, the manufacturing computer 156 produces machine-readable code 166 for operating digitally controlled manufacturing equipment 22 to produce the exemplary appliance(s) 60, 172. The machine-readable code 166 may be based on the prescription 62 and, as such, may include all or any single one of the commands 82, 112 found in the prescription 62. For example, the orthodontist may build the prescription 62 with a Use TruGen XR command 82 in which case the machine-readable code 166 based on the prescription 62 may incorporate the TruGen XR command verbatim from the prescription 62.

For manufacture of orthodontic appliances, the manufacturing equipment 22 preferably includes forming machinery 170 which produces the appliances, such as orthodontic brackets 172 themselves, or molds for the appliance 60. Automated bracket or mold making can be carried out by casting or molding of the brackets from molds made by the automated machines, by cutting slots at calculated angles or machining other features in preformed blanks, such as with CNC machinery 174, or by other automated bracket making methods. The machine 170 may shape the surfaces of preformed bracket bases, providing a design option of torquing the teeth by either the bracket slot or base, as may be best for various bracket materials. The equipment 170 may also include an appliance archwire bending machine or other type of wire forming machine to produce custom shaped archwires for the appliance 172.

With reference to FIG. 14, as a result, the aligner 60 includes a hollow shell 200 that is configured to encapsulate one or more crowns of a patient's teeth. The shell 200 is formed with a plurality of cavities 202 that collectively define an edge 204, which defines an opening 206. Each cavity 202 is shaped to receive a specific one of the patient's teeth through the opening 206 during use of the aligner 60. The shell 200 is made of an elastic material in one or more layers and may include one or more receptacles 208 that are configured to receive an attachment (not shown) on the patient's tooth and/or one or more devices in the aligner 60. In that regard, the library 84 may include a command 82 to add an attachment to a specified tooth in the prescription 62. The appliance(s) are shipped to the orthodontist 18 or the patient 30 for orthodontic treatment.

During orthodontic treatment, the aligner 60 is selectively positioned over the patient's teeth and may fit tightly due to slight differences in the position of one or more of the cavities 202 relative to the corresponding tooth. A forcible contact with the aligner 60 may move the patient's teeth toward a predetermined position according to a patient's treatment plan that may ultimately end at T2. A set of aligners (not shown) may include one or more aligners 60. During orthodontic treatment, each stage of treatment may include an aligner that progressively moves one or more of the patient's teeth incrementally toward a desired final arrangement. The individual aligners are utilized in a predetermined sequence according to the treatment plan approved by the orthodontist 18 to complete orthodontic treatment or move the patient's teeth to T2. Accordingly, each aligner in the series may move one or more teeth a prescribed amount. While similar, each aligner is slightly different in shape. Cumulatively, these individual amounts may result in complete treatment of the patient's malocclusion.

In general, the routines and instructions executed to implement the embodiments of the invention, whether implemented as part of an operating system or a specific application, component, program, object, module or sequence of instructions, or a subset thereof, may be referred to herein as “computer program code,” or simply “program code.” Program code typically comprises computer-readable instructions that are resident at various times in various memory and storage devices in a computer, such as, any one of or a combination of computers 38, 152, 154, 156 or in the appliance manufacturing equipment 22 and that, when read and executed by one or more processors in a computer, cause that computer to perform the operations necessary to execute operations and/or elements embodying the various aspects of the embodiments of the invention. Computer-readable program instructions for carrying out operations of the embodiments of the invention, such as the arrangement of elements in the interface 64, 78 and display of interface 64, 78 on display 52 may be, for example, assembly language or either source code or object code written in any combination of one or more programming languages.

Various program code described herein may be identified based upon the application within which it is implemented in specific embodiments of the invention. However, it should be appreciated that any particular program nomenclature which follows is used merely for convenience, and thus the invention should not be limited to use solely in any specific application identified and/or implied by such nomenclature. Furthermore, given the generally endless number of manners in which computer programs may be organized into routines, procedures, methods, modules, objects, and the like, as well as the various manners in which program functionality may be allocated among various software layers that are resident within a typical computer (e.g., operating systems, libraries, API's, applications, applets, etc.), it should be appreciated that the embodiments of the invention are not limited to the specific organization and allocation of program functionality described herein.

The program code embodied in any of the applications/modules described herein, such as, the prescription 62 of commands 82, 112 or the commands 82, 112 themselves is capable of being individually or collectively distributed as a program product in a variety of different forms. In particular, the program code may be distributed using available means for distribution, including direct download from an internet accessible computer or via a computer-readable storage medium having computer-readable program instructions thereon for causing a processor to carry out aspects of the embodiments of the invention.

Computer-readable storage media, which is inherently non-transitory, may include volatile and non-volatile, and removable and non-removable tangible media implemented in any method or technology for storage of data, such as computer-readable instructions, data structures (e.g., imagery 24, 3-D digital model 54 and 56, prescription 62, user interfaces 52, 78, library 84), program modules, or other data. Computer-readable storage media may further include RAM, ROM, erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired data and which can be read by a computer. A computer-readable storage medium should not be construed as transitory signals per se (e.g., radio waves or other propagating electromagnetic waves, electromagnetic waves propagating through a transmission media such as a waveguide, or electrical signals transmitted through a wire). Computer-readable program instructions may be downloaded to a computer, another type of programmable data processing apparatus, or another device from a computer-readable storage medium or to an external computer or external storage device via a network.

Computer-readable program instructions stored in a computer-readable medium may be used to direct a computer, other types of programmable data processing apparatuses, or other devices to function in a particular manner, such that the instructions stored in the computer-readable medium produce an orthodontic appliance including instructions that implement the functions, acts, and/or operations specified in flow-charts, sequence diagram, and/or block diagrams. The computer program instructions may be provided to one or more processors of a general-purpose computer, a special-purpose computer, or other programmable data processing apparatus to produce a prescription and/or an appliance, such that the instructions, which execute via the one or more processors, cause a series of computations to be performed to implement the functions, acts, and/or operations specified in the flow-charts, sequence diagrams, and/or block diagrams.

### EXAMPLES

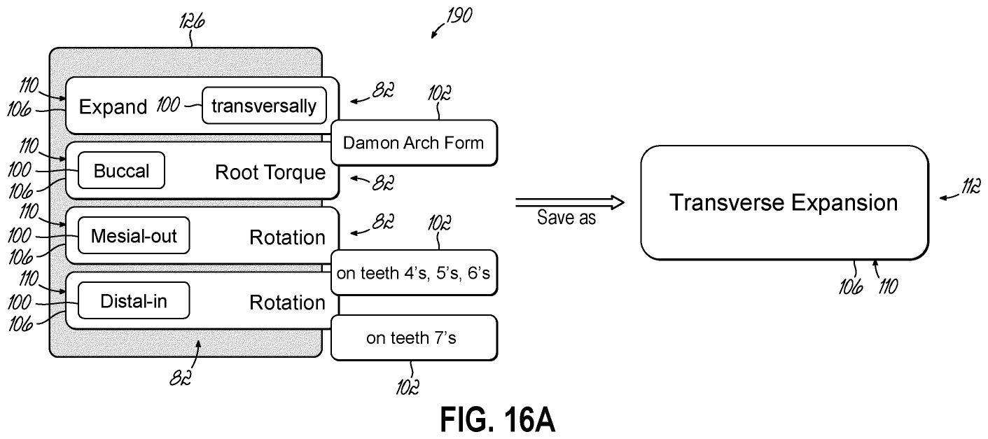

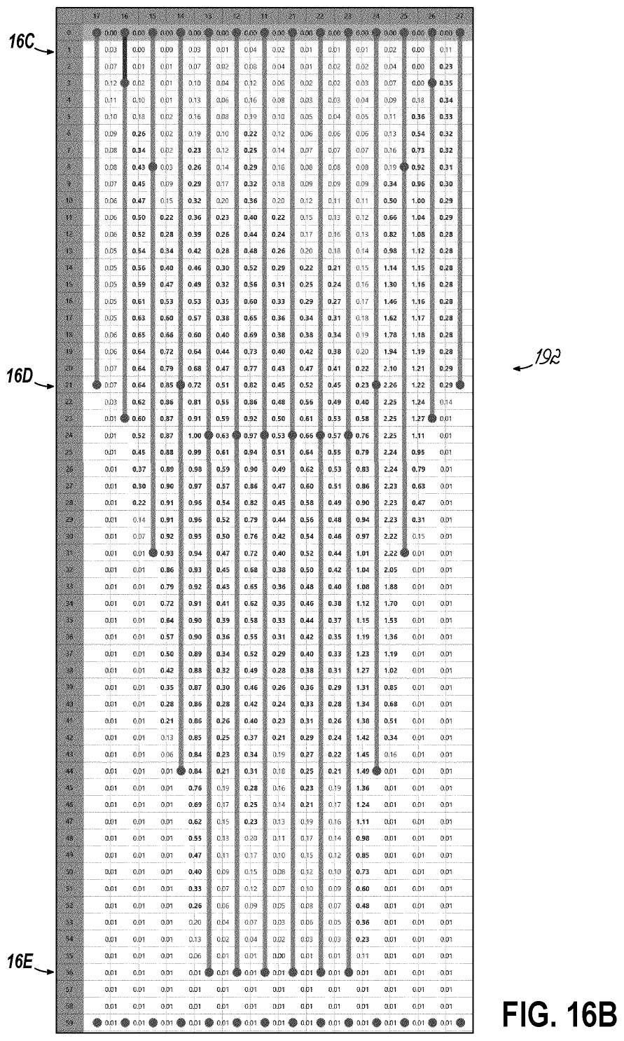

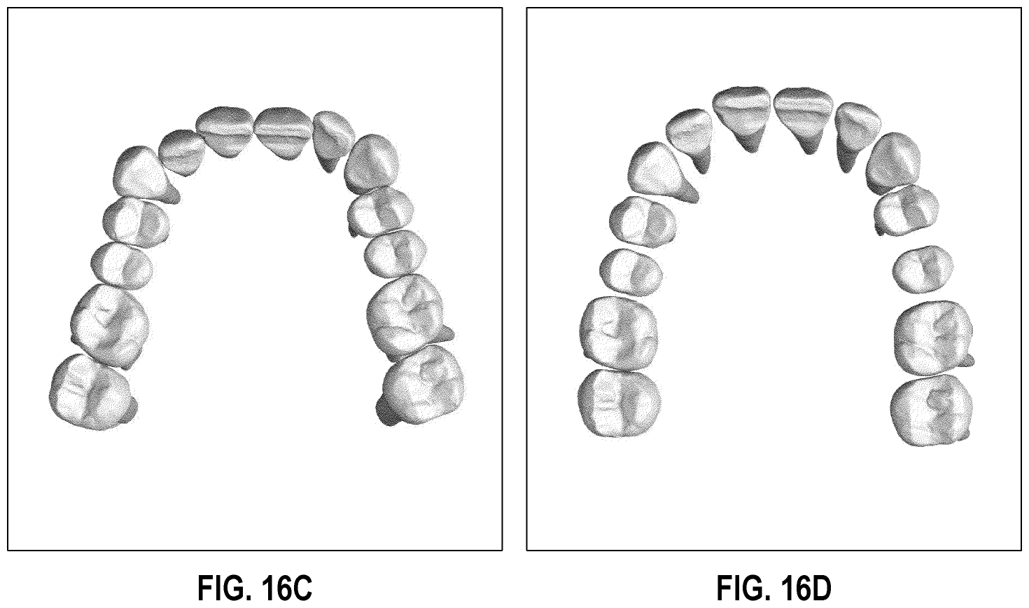

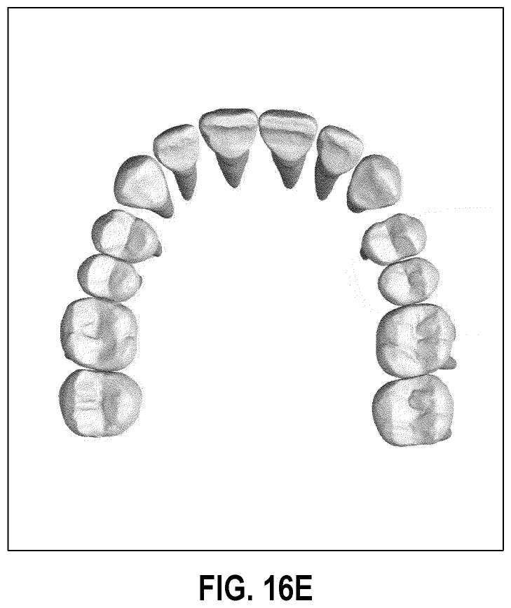

A prophetic example of embodiments of the invention is shown by reference to FIGS. 16A, 16B, 16C, 16D, and 16E. In FIG. 16A, there is a prescription 190 developed through the interface 64, 78 of the system 10. The prescription 190 is built based on an orthodontist's diagnosis of T1. As shown, the commands are block-based 110 and are stacked with each of Expand, Root Torque, Rotation, and Rotation commands 82 being wrapped 126. Thus, these four commands 82 are to be applied simultaneously. In further detail, Expand 82 is transversally 100 with a Damon Arch Form 102. Root Torque 82 is Buccal 100. Rotation 82 is Mesial-out 100 on teeth 4, 5, 6 102 (according to the FDI teeth number system). A second Rotation 82 is Distal-In 100 on teeth 7 102. The prescription 190 is a construction of basic commands 82 saved as a user-defined command 112 of “Transverse Expansion” and determines the overall movement of teeth depicted by a staging plan 192 (FIG. 16B) in a treatment plan of the patient for application of aligners.

In FIG. 16B, the left side of the staging plan 192 shows the stage number 0 (beginning of treatment, the T1 model) to stage 56 (end of treatment, T2 model). The top of the plan 192 lists the individual's teeth (according to FDI notation) 17-11 and 21-27. The T2 model is at stage 56 and is shown in FIG. 16E. At each stage (row), the values (e.g., 0.00, 0.01, etc.) represent the space in millimeters between the indicated, adjacent teeth at the top of the staging plan 192. For example, between tooth 16 and tooth 15 at stage 6 there is a 0.26 mm space. Solid vertical lines with filled-in circles that extend from one stage and cross over one or more stages indicate tooth movement from the stage indicated to the lower stage indicated. For example, all teeth begin moving at stage 1 with tooth 17 stopping movement at stage 21, which is that tooth's position in the T2 model, while tooth 12 moves until stage 56, where it reaches it's final position in the T2 model.

In this example and according to the prescription 190, the staging plan 192 shows all teeth moving at stage 1 because all commands are simultaneously applied with a Expand command. As shown by way of comparison between FIG. 16C and FIG. 16D, Expand and Root Torque are applied with Rotation being applied to selected teeth. Expansion is shown in the increase in the spaces between the teeth in the staging plan 192 between stage 1 and stage 21. For example, the space between teeth 14 and 13 is 0.03 mm at stage 1 (FIG. 16C). This space gradually increases to 0.72 mm at stage 21 (FIG. 16B). According to the staging chart 192 at stage 21 (FIG. 16D), the posterior teeth complete their movement. From stage 21 to stage 56, the spaces between the anterior teeth are substantially eliminated at stage 56 (FIG. 16E).

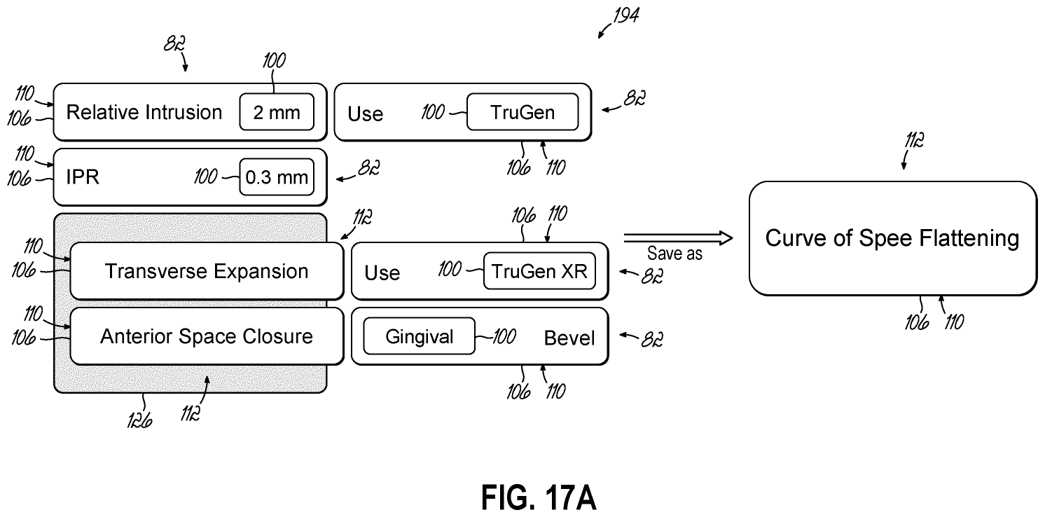

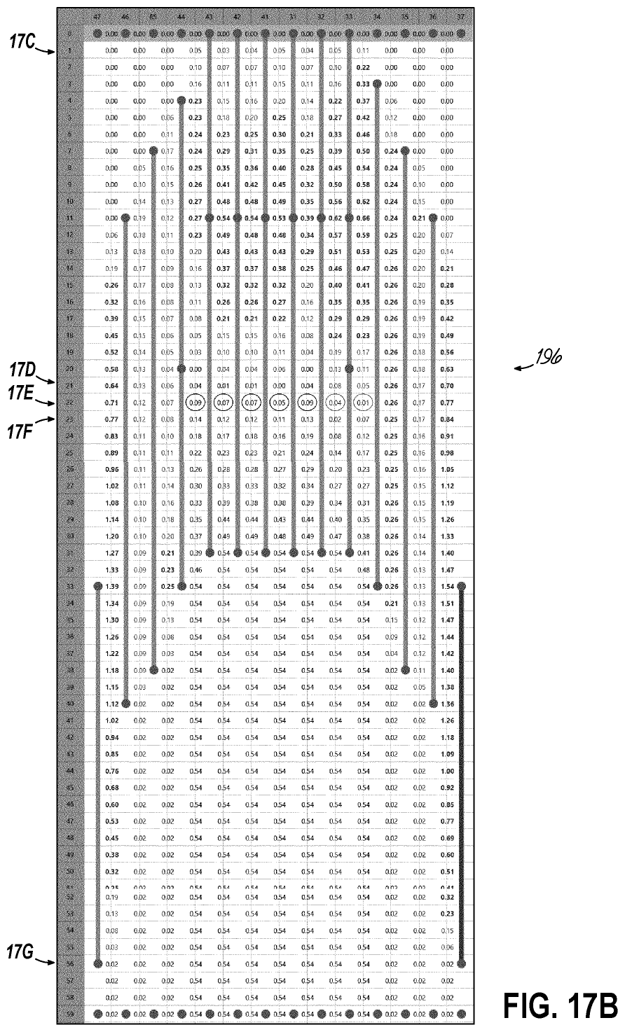

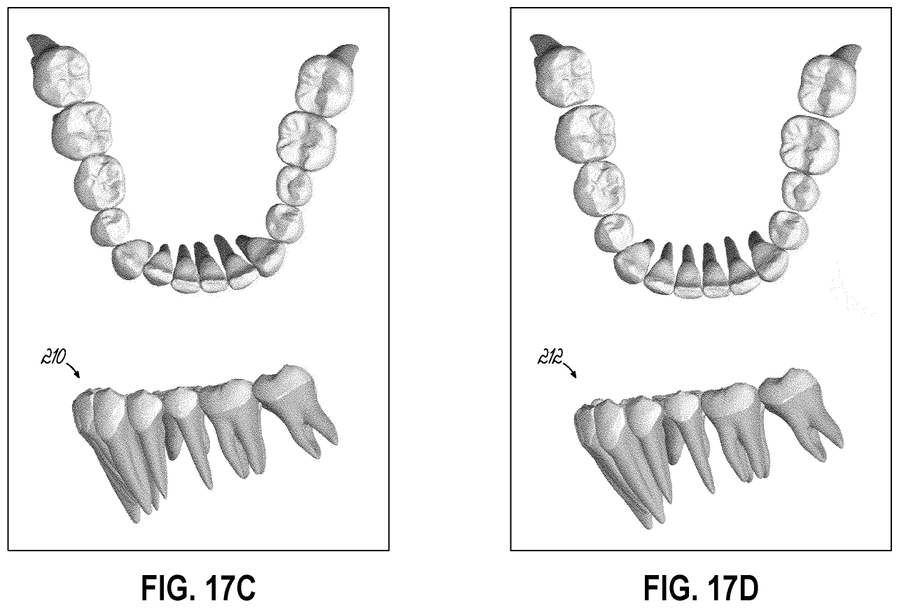

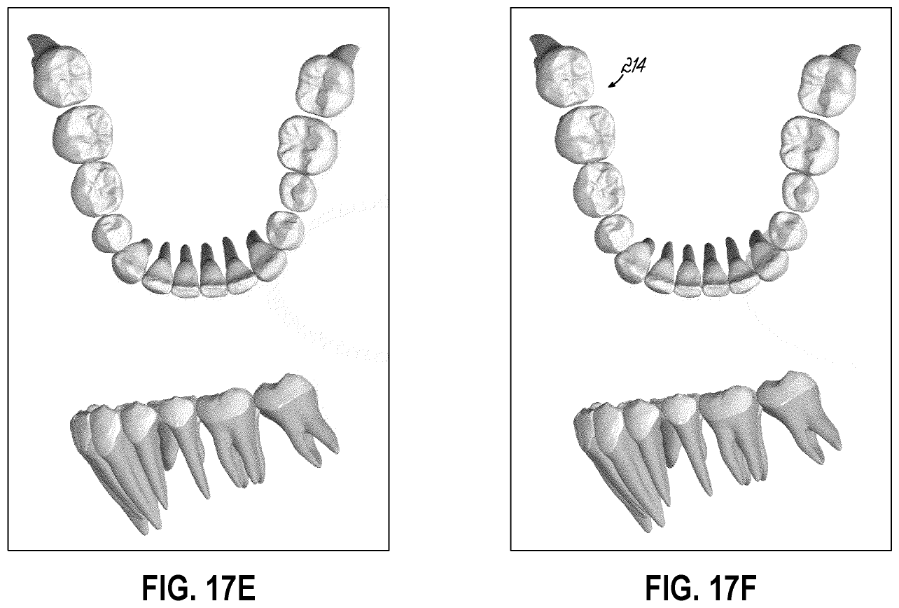

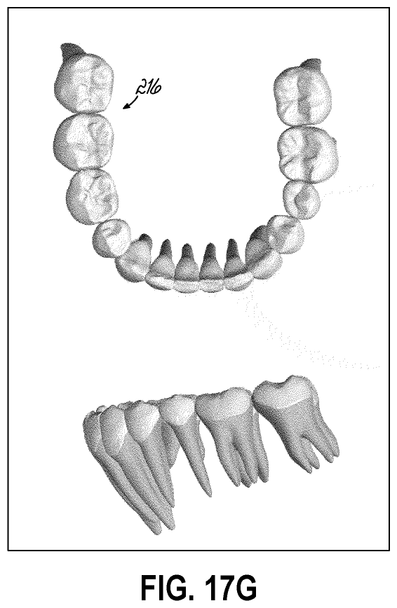

A second prophetic example of embodiments of the invention is shown by reference to FIGS. 17A, 17B, 17C, 17D, 17E, 17F, and 17G. In FIG. 17A, there is a prescription 194 developed through the interface 64, 78 of the system 10. The prescription 194 is built based an orthodontist's diagnosis of T1. The commands are block-based 110 and are stacked with Relative Intrusion 82 of 2 mm 100 as modified with Use 82 TruGen 100 being applied first. IPR 82 of 0.3 mm 100 is to be applied second. Transverse Expansion 112 as modified with Use 82 TruGen XR 100 and Anterior Space Closure 112 as modified by Bevel 82 in Gingival 100 are wrapped 126 indicating their simultaneous application. The Anterior Space Closure and Transverse Expansion commands are themselves user-defined commands 112, the basic commands for which are shown in FIGS. 10 and 11, respectively. Following IPR, each of Transverse Expansion 112 and Anterior Space Closure 112 are to be applied simultaneously. The prescription 194 is a construction of basic commands 82 and user-defined commands 112 determines the overall movement of teeth provided by a staging plan 196 (FIG. 17B) in a treatment plan of the patient for application of aligners.

In FIG. 17B, the left side of the staging plan 196 shows the consecutive stage numbers during treatment. Stage 0 represents the T1 model. The last stage, stage 56, is the end of treatment and represents the T2 model (shown in FIG. 17G). The top of the staging plan 196 lists the individual's teeth 47-41 and 31-37 (according to FDI notation). At each stage (row), the values (e.g., 0.00, 0.05, etc.) represent the space in millimeters between the indicated, adjacent teeth at the top of the staging plan 196. For example, between tooth 47 and tooth 46 at stage 12 there is a 0.06 mm space. Solid vertical lines with filled-in circles that extend from one stage and cross over one or more stages indicate tooth movement from the stage indicated and to the lower stage indicated. For example, tooth 44 begins moving at stage 4 and moves during each stage until stage 33, where it reaches it's final position in the T2 model.

In this example and according to the prescription 194, the staging plan 196 shows that the anterior teeth (43, 42, 41, 31, 32, 33) are moved first, with the vertical lines starting from stage 1. This movement is illustrated in the teeth depicted in FIGS. 17B, 17C, and 17D. Tooth movement according to the Relative Intrusion command is shown in accordance the order specified in the prescription of FIG. 17A. FIG. 17C represents the teeth positions at stage 1 of the staging plan 196 (FIG. 16B). FIG. 17D represents the teeth positions at stage 21 of the plan 196. Comparison of the bottom model at arrow 210 relative to arrow 212 in each of FIG. 17C and FIG. 17D illustrates intrusion of the anterior teeth according to the Relative Intrude command. Stage 21 is before IPR of the anterior teeth 43-41 and 31-33 (indicated by circled values in FIG. 17B).

Once the intrusion of the anterior teeth reaches 2 mm, IPR in accordance with the IPR command in the prescription 194 is applied. FIGS. 17D, 17E, and 17F illustrate IPR as indicated in stage 22 (FIG. 17E) applied between teeth 44-43, 43-42, 42-41, 41-31, and 31-32. FIGS. 17F and 17G illustrate simultaneous application of the Transverse Expansion and Anterior Space Closure commands by which the posterior teeth are moving together. Posterior teeth 47 and 37 move the most during this period of treatment and is shown generally at arrow 214 of FIG. 17F relative to arrow 216 in FIG. 17G as the space between the teeth at this location has closed. The elimination of this space is also shown in FIG. 16B.

While the present invention has been illustrated by a description of various embodiments and while these embodiments have been described in some detail, it is not the intention of the inventors to restrict or in any way limit the scope of the appended claims to such detail. Thus, additional advantages and modifications will readily appear to those of ordinary skill in the art. The various features of the invention may be used alone or in any combination depending on the needs and preferences of the user.

What is claimed is:

1. A system for orthodontic treatment planning for a patient, the system comprising:
a processor;
memory coupled to the processor, the memory configured to store computer-program instructions that, when executed by the processor cause the system to:
display a user interface on a display, the user interface for a user to input a prescription for treatment of the patient, the user interface providing a plurality of commands for selection, wherein each command is a predetermined instruction based on orthodontic nomenclature for moving or modifying one or more of a patient's teeth; and
receive a selected two or more of the plurality of commands into the prescription for treatment, wherein the two or more selected commands are to be applied according to a predetermined protocol to the patient's teeth.

2. The system of claim 1, further comprising:
a database coupled to the memory and accessible by the processor, the database being configured to receive the prescription for treatment of the patient and to contain a plurality of other prescriptions for treatment of other patients.

3. The system of claim 2, wherein the database is configured to receive a plurality of other prescriptions from a plurality of users of the system.

4. The system of claim 1, wherein the predetermined instruction is selected from a one-word instruction, a two-word instruction, and a three-word instruction or a combination thereof.

5. The system of claim 1, wherein the predetermined instruction is selected from the group consisting of a one-word instruction, a two-word instruction, and a three-word instruction.

6. The system of claim 1, wherein when executed by the processor, the computer-program instructions cause the system to:
limit a selected one of the plurality of commands to a single line in the prescription for treatment.

7. The system of claim 1, wherein when executed by the processor, the computer-program instructions cause the system to:
identify the two or more selected commands in the prescription for treatment for simultaneous application to the patient's teeth according to the predetermined protocol.

8. The system of claim 1, wherein when executed by the processor, the computer-program instructions cause the system to:
arrange the two or more selected commands in the prescription for treatment for sequential application to the patient's teeth according to the predetermined protocol.

9. The system of claim 1, wherein when executed by the processor, the computer-program instructions cause the system to:
arrange the two or more selected commands in the prescription for treatment for sequential application to the patient's teeth according to the predetermined protocol; and
identify at least one of the two or more selected commands for sequential application for simultaneous application with at least one other of the two or more selected commands to the patient's teeth according to the predetermined protocol.

10. The system of claim 1, wherein when executed by the processor, the computer-program instructions cause the system to:
prevent entry of text into the prescription for treatment that is not one of the selected commands.

11. The system of claim 1, wherein when executed by the processor, the computer-program instructions cause the system to:
display, in the user interface, a rectangular-shaped border encircling the predetermined instruction of each command.

12. The system of claim 11, wherein when executed by the processor, the computer-program instructions cause the system to:
stack two or more rectangular-shaped borders one above the next in the user interface.

13. The system of claim 12, wherein when executed by the processor, the computer-program instructions cause the system to:
save a combination of the stacked two or more selected commands as a user-defined command to the memory.

14. The system of claim 1, wherein when executed by the processor, the computer-program instructions cause the system to:
save the prescription for treatment in a machine-readable format.

15. The system of claim 1, wherein when executed by the processor, the computer-program instructions cause the system to:
generate a staging plan based on the prescription for treatment for review by an orthodontist.

16. The system of claim 1, further comprising:
appliance manufacturing equipment configured to manufacture an appliance based on the prescription for treatment, wherein when executed by the processor, the computer-program instructions cause the system to:
transmit the prescription for treatment to the appliance manufacturing equipment and the appliance manufacturing equipment reads the prescription for treatment.

17. A system for building an orthodontic treatment plan applicable to teeth of a patient, the system comprising:
a user interface for interfacing with a computer program;
the computer program being configured to interact with a user through the user interface to:
display a representation of the teeth of the patient, wherein the user selects one or more teeth from the representation of the teeth of the patient for treatment according to the orthodontic treatment plan; and
display a plurality of commands from a library of commands, wherein the library of commands is predetermined, the displayed commands are predetermined instructions based on orthodontic nomenclature for moving the selected one or more of the teeth to a new position, and the user selects one or more of the plurality of commands for inclusion in the orthodontic treatment plan.

18. The system of claim 17, wherein displaying the plurality of commands, the computer program is further configured to:
retrieve an initial position of the selected one or more teeth;
determine a final position of the tooth subsequent to orthodontic treatment; and
determine a staging plan for moving the tooth from the initial position to the final position based on the selected one or more of the plurality of commands in the orthodontic treatment plan.

19. The system of claim 17, wherein each predetermined command is selected from a one-word instruction, a two-word instruction, and a three-word instruction or a combination thereof.

20. A computer-implemented method of creating an orthodontic treatment plan applicable to teeth of a patient, comprising:
receiving a digital model of a patient's teeth in a first arrangement;
selecting one or more commands from a library of commands, wherein the library of commands is predetermined and the selected commands are based on orthodontic nomenclature for moving or modifying one or more of the patient's teeth;
placing the selected one or more commands into a prescription for orthodontic treatment of the patient;
converting the prescription into machine-readable code for use by a processor of a computer; and
creating a second digital model of the patient's teeth in a second arrangement different from the first arrangement based on orthodontic treatment according to the machine-readable code.

21. The computer-implemented method of claim 20, further comprising:
determining a staging plan for moving or modifying the one or more of the patient's teeth from positions in the first model to positions in the second model based on the prescription for orthodontic treatment of the patient.

22. The computer-implemented method of claim 20, wherein each command is a one-word instruction, a two-word instruction, or a three-word instruction.

23. A computer-implemented method of building an orthodontic treatment plan applicable to teeth of a patient, the method comprising:
displaying a first digital model of the teeth of the patient to a user;
receiving from the user a selection of one or more teeth of the first digital model;
displaying a plurality of commands from a library of commands, wherein the library of commands is predetermined and the displayed commands are instructions based on orthodontic nomenclature for manipulating the selected one or more of the teeth, each command manipulating the teeth in a distinct way from each other command;
receiving from the user a selection of the one or more displayed commands, wherein the selected commands make up the orthodontic treatment plan; and
creating a second digital model of the teeth of the patient by moving and/or modifying at least selected one or more teeth of the first digital model based on the orthodontic treatment plan.

24. The computer-implemented method of claim 23, wherein receiving a selection from the user of the one or more displayed commands includes the user selecting a one-word instruction, a two-word instruction, or a three-word instruction or a combination thereof from the predetermined library of commands, and the creating the second digital model of the teeth includes applying the selected command to the selected one or more teeth.

25. The computer-implemented method of claim 23 further comprising, after creating, displaying the created second digital model of the teeth of the patient to the user.

26. The computer-implemented method of claim 23 further comprising, after creating, designing a mold using the second digital model, wherein the mold is usable in manufacturing an orthodontic treatment device.

27. A method of preparing a prescription for orthodontic treatment comprising:
selecting two or more commands from a library of commands, wherein the library of commands is predetermined and the selected command is a one-word instruction, a two-word instruction, or a three-word instruction based on orthodontic nomenclature for moving or modifying one or more of a patient's teeth; and
placing the selected two or more commands in a predetermined order of application into a prescription for orthodontic treatment of the patient.

28. The method of claim 27, wherein placing the selected commands includes placing a first command on a first line of the prescription and a second command on a second line of the prescription.

29. The method of claim 28, wherein the predetermined order of application is the first command first and the second command after the first command.

30. The method of claim 27, wherein placing includes grouping the first command and the second command.

31. The method of claim 30, wherein the predetermined order of application is simultaneous application of the first command and the second command.

32. The method of claim 27, wherein placing the selected commands includes placing a first command on a first line of the prescription and a second command on a second line of the prescription and wherein the method further comprising:
selecting a third command;
placing the third command on a third line of the prescription;
grouping the first command and the second command or the second command and the third command for simultaneous application to the patient's teeth according to the predetermined protocol; and
arranging the first command or the third command for sequential application with the grouped commands to the patient's teeth according to the predetermined protocol.

33. The method of claim 27, wherein at least one of the two or more commands include a user-defined variable, and wherein following selecting two or more commands, the method further comprises:
modifying the user-defined variable of the selected command.
